(19) United States
(12) Patent Application Publication
(10) Pub. No.: US 2009/0297549 A1
(43) Pub. Date: Dec. 3, 2009

(54) CONSERVED AND SPECIFIC STREPTOCOCCAL GENOMES

(76) Inventors: Herve Tettelin, Rockville, MD (US); Vega Masignani, Panteneto (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/468,930

(22) Filed: May 20, 2009

Related U.S. Application Data
(63) Continuation of application No. 10/525,536, filed on Jul. 19, 2006, now abandoned.
(60) Provisional application No. 60/406,237, filed on Aug. 26, 2002.
(60) Provisional application No. 60/406,676, filed on Aug. 27, 2002.
(60) Provisional application No. 60/406,757, filed on Aug. 28, 2002.

(51) Int. Cl. A61K 39/09; C07H 21/04; A61P 31/04

(52) U.S. Cl. 424/190.1; 536/23.1

(57) ABSTRACT

The invention relates to polynucleotides which are conserved or specific to one or more species of Streptococcus, Streptococcus species serotypes, and/or serotype isolates. In particular, the invention relates to polynucleotides from Streptococcus which are conserved or specific to one or more of the species of S. pneumoniae (“pneumococcus” or “S. pn.”), S. pyogenes (“group A streptococcus” or “GAS”), and S. agalactiae (“group B streptococcus” or “GBS”). The invention further relates to polynucleotides which are conserved or specific to one or more Streptococcal species serotypes, such as GBS serotypes Ia, Ib, II, III, IV, V, VI, VII, and VIII. The invention still further relates to polynucleotides which are conserved or specific to one or more clinical isolates of a Streptococcus species.

### CROSS REFERENCE TO RELATED APPLICATIONS

This application is a continuation of Ser. No. 10/525,536 filed on Aug. 26, 2003, which claims priority of provisional patent application Ser. Nos. 60/406,237 filed on Aug. 26, 2002; 60/406,676 filed on Aug. 27, 2002; and 60/406,757 filed on Aug. 28, 2002. Each of these applications is incorporated by reference in its entirety herein.

This application incorporates by reference the contents of a 2.59 MB text file created on May 20, 2005 and named “51227sequencelisting.txt,” which is the sequence listing for this application.

### FIELD OF THE INVENTION

The invention relates to polynucleotides which are conserved or specific to one or more species of Streptococcus, Streptococcus species serotypes, and/or serotype isolates. The conserved or specific genomic regions can be used to identify, screen and develop vaccines and other treatments for Streptococcal infections and can be used in diagnostic assays to diagnose and identify Streptococcal infections.

### BACKGROUND OF THE INVENTION

The genus Streptococcus consists of Gram-positive, chain-forming, spherical bacterial cells. Three species of clinical interest are S. pneumoniae (“pneumococcus” or “S.pn.”), S. pyogenes (‘group A streptococcus’ or ‘GAS’) and S. agalactiae (‘group B streptococcus’ or ‘GBS’). Infections with these three pathogenic streptococci lead to conditions including pharyngitis, toxic shock syndrome and necrotizing fasciitis.

Once thought to infect only cows, GBS is now known to cause serious disease, bacteraemia and meningitis in immunocompromised individuals and neonates. There are two known types of neonatal infection. The first (early onset, usually within 5 days of birth) is manifested by bacteraemia and infection. It is generally contracted vertically as a baby passes through the birth canal. GBS is thought to colonize the vagina of about 25% of young women; approximately 1% of infants born via a vaginal birth to colonised mothers will become infected. Mortality resulting from these infections is between 50-70%. The second type of neonatal infection is a meningitis that occurs 10 to 60 days after birth. If pregnant women are vaccinated with type III capsule so that the infants are passively immunised, the incidence of the late onset meningitis is generally reduced, although not entirely eliminated.

The “B” in “GBS” refers to the Lancefield classification, which is based on the antigenicity of a carbohydrate which is soluble in dilute acid and called the C carbohydrate. Lancefield identified 13 types of C carbohydrate, designated A to O, that could be serologically differentiated. The organisms that most commonly infect humans are found in groups A, B, D, and G. Within group B, strains can be divided into at least 9 serotypes (Ia, Ib, II, III, IV, V, VI, VII, and VIII) based on the structure of their polysaccharide capsule. Further categories based on, for example, the expression of certain proteins have also been developed.

GBS strains of polysaccharide capsule Type V were rarely isolated before the mid-1980's but now account for approximately one-third of clinical isolates in the US. Type V is the most common capsular serotype associated with invasive infection in nonpregnant adults, and the emergence of Type V strain over the past decade has been temporarily linked to an increase in GBS disease in this population.

Group A streptococcus is a frequent human pathogen, estimated to be present in between 5-15% of normal individuals without signs of disease. When host defenses are compromised, or when the organism is able to exert its virulence, or when it is introduced into vulnerable tissues or hosts, however, an acute infection occurs. Diseases include puerperal fever, scarlet fever, erysipelas, pharyngitis, impetigo, necrotising fasciitis, myositis and streptococcal toxic shock syndrome.

Pneumococcus is the most common cause of acute respiratory infection and otitis media and is estimated to result in over 3 million deaths in children every year worldwide from pneumonia, bacteremia, or meningitis. Even more deaths occur among elderly people, among whom S. pn. is the leading cause of community-acquired pneumonia and meningitis. Since 1990, the number of penicillin-resistant strains has increased from 1 to 5% to 25 to 80% of isolates, and many strains are now resistant to commonly prescribed antibiotics such as penicillin, macrolides, and fluoroquinolones. See Tettelin, et al. (2001) Science 293, 248-506.

The complete genomic sequence of a virulent isolate of S. pneumoniae was published by Tettelin, et al. (2001) Science 293, 248-506 and is available at the TIGR website at http://www.tigr.org. as well as on GEN BANK (available through the Pub Med website at http://www.ncbi.nlm.nib.gov/entrez/queuery.fcgi). The genomic sequence, the Tettelin article and its published supplemental material are incorporated herein by reference in their entirety.

The complete genomic sequence of an Ml strain of S. pyrogenes was published by Ferretti, et al. (2001) Proc. Natl. Acad. Sci. USA 98, 4658-4663 and is available at the TIGR website at http://www.tigr.org. The genomic sequence, the Ferretti article and its published supplemental materials are incorporated herein by reference in their entirety.

The complete genomic sequence of a serotype V strain of S. agalactiae (type V strain 2603 V/R) was published on Aug. 28, 2002 at Gen Bank Accession no. AE009948 (available through Pub Med at http://www.ncbi.nlm.nih.gov/entrez/query.fcgi and/or was available on the same day at the TIGR website at http://www.tigr.org. Most of this sequence is also available in PCT International Patent Application Publication WO 02/34771. The genomic sequence, the Tettelin article and its published supplemental materials are incorporated herein by reference in their entirety.

Current treatments for Streptococcal infections include both antibiotics and prophylactic vaccination. Current vaccines, particularly with respect to GBS, suffer from poor immunogenicity, while the emergence of antibiotic resistant strains has lessened the effectiveness of currently used antibiotics. Accordingly, there is an increasing need for the development of new vaccines and antibiotics (as well as other small molecule bacterial inhibitors) to help prevent and treat Streptococcal infections.

Applicants have identified regions of the Streptococcal genomes which can be used to identify and develop new vaccines and treatments for Streptococcal infections. Specifically, Applicants have identified polynucleotides of the Streptococcal genome which are conserved or specific to Streptococcal species, species serotypes, and/or specific serotype isolates. These polynucleotides and their expressed polypeptides can be used to screen, develop and design new vaccines, antibiotics and other small molecule bacterial inhibitors. These polynucleotides and their expressed polypeptides can further be used to diagnose and identify Streptococcal infections.

### SUMMARY OF THE INVENTION

The invention relates to polynucleotides which are conserved or specific to one or more species of Streptococcus, Streptococcus species serotypes, and/or serotype isolates. In particular, the invention relates to polynucleotides from Streptococcus which are conserved or specific to one or more of the species of S. pneumoniae (“pneumococcus” or “S. pn.”), S. pyogenes (“group A streptococcus” or “GAS”), and S. agalactiae (“group B streptococcus” or “GBS”). The invention further relates to polynucleotides which are conserved or specific to one or more Streptococcal species serotypes, such as GBS serotypes Ia, Ib, II, III, IV, V, VI, VII, and VIII. The invention still further relates to polynucleotides which are conserved or specific to one or more clinical isolates of a Streptococcus species.

The invention is based on the identification of the following Subsets of genes. Genes falling within each subset are described with respect to referenced tables, lists, and/or figures (in particular the CGH map depicted in FIG. 1).

The following Subsets relate to the GBS genome:

GBS Subset 1: 1060 GBS genes which have homologs with GAS and with pneumococcus (Table 8);

GBS Subset 2: 225 GBS genes which have homologues with GAS, but not with pneumococcus (Table 10);

GBS Subset 3: 176 GBS genes which have homologues with pneumococcus but not with GAS (Table 9);

GBS Subset 4: 683 GBS genes which do not have homologues with GAS or pneumococcus (specific to GBS vs GAS and pneumococcus) (Table 11).

The invention is based on the identification of the following subsets of genes within the GAS genome:

GAS Subset 1: 1006 GAS genes which have homologues with GBS and with pneumococcus (Table 33);

GAS Subset 2: 212 GAS genes which have homologues with GBS but do not have homologues with pneumococcus (Table 34);

GAS Subset 3: 62 GAS genes which have homologues with pneumococcus but do not have homologues with GBS (Table 35);

GAS Subset 4: 416 GAS genes which do not have homologues with either GBS or pneumococcus. This Subset can be determined by subtracting the above subsets from the published genome.

The invention is based on the identification of the following subsets of genes within the pneumococcus genome:

Spn Subset 1: 1034 Spn genes which have homologues with GBS and GAS (Table 36);

Spn Subset 2: 195 Spn genes which have homologues with GBS but do not have homologues with GAS (Table 37);

Spn Subset 3: 74 Spn genes which have homologues with GAS but do not have homologues with GBS (Table 38);

Spn Subset 4: 836 Spn genes which do not have homologues with either GBS or pneumococcus. This Subset can be determined by subtracting the above Subsets from the published genome.

The invention further provides polynucleotides which are conserved or specific to Streptococcus based on a comparison with a wide range of published bacterial genomes. The following additional Subsets are provided:

GBS Subset 1(a): Of the 1060 GBS genes which have homologues in both GAS and pneumococcus, 12 of those GBS genes do not have homologues with any of the other published bacterial genomes at the time of the invention (i.e., GBS Subset 1(a) is specific to Streptococcus vs non Streptococcus published genomes). (The 12 GBS ORF's are listed in Table 3).

GBS Subset 2(a): This Subset comprises GBS genes which have homologues with GAS, but not with pneumococcus or any other published bacterial genomes at the time of the invention.

GBS Subset 3(a): This Subset comprises GBS genes which have homologues with pneumococcus, but not with GAS or any other published bacterial genomes at the time of the invention.

GBS Subset 4(a): Of the 683 GBS genes which do not have homologues in either GAS or pnuemococcus, 315 of these GBS genes also do not have homologues with any of the other published bacterial genomes. These include six proteins predicted to be anchored on the cell wall (SAG0677, SAG0771, SAG1052, SAG1331, SAG1473, and SAG1168), three of the capsule-related genes (SAG1163, SAG1167, and SAG1168), six transcriptional regulators, and four genes of the cyl operon (SAG0663-SAG0673) essential for GBS hemolytic activity and production of pigment. See Pritzlaff et al. (2001) Mol. Microbiol., 39, 236-247. The rest of the 315 proteins include 240 hypothetical proteins with no similarity to other proteins in databases.

Many of the 315 genes specific to S. agalactiae are located in regions likely to constitute mobile genetic elements. Two of these regions resemble prophages (SAG0545-SAG0610 and SAG1835-SAG1885) displaying a mosaic structure with segments most similar to different bacteriophages, a pattern that suggests frequent recombination events. PblA and PblB are adhesins from a S. mitis prophage where they contribute to endocarditis by binding to human platelets (See Bensing, et al. (2001) Infect. Immun. 69, 6186-6192; Bensing, et al (2001) Infect. Immun. 69, 1373-1380. Their orthologs in S. agalactiae are located on separate prophages and display a different protein structure. Another region (SAG1247-SAG1299) encodes a putative conjugative transposon that carries genes for cadmium efflux and mercury resistance.

GAS Subset 1(a): This Subset comprises GAS genes which have homologues with GBS and with pneumococcus, but do not have homologues with any of the other published bacterial genomes at the time of the invention.

GAS Subset 2(a): This Subset comprises GAS genes which have homologues with GBS but do not have homologues with pneumococcus or any of the other published bacterial genomes at the time of the invention;

GAS Subset 3(a): This Subset comprises GAS genes which have homologues with pneumococcus but do not have homologues with GBS or any of the other published bacterial genomes at the time of the invention.

GAS Subset 4(a): This Subset comprises GAS genes which do not have homologues with either GBS or pneumococcus or with any of the other published bacterial genomes at the time of the invention.

Spn Subset 1(a): This Subset comprises Spn genes which have homologues with GBS and GAS but which do not have homologues with any of the other published bacterial genomes at the time of the invention;

Spn Subset 2(a): This Subset comprises Spn genes which have homologues with GBS but do not have homologues with GAS or with any of the other published bacterial genomes at the time of the invention;

Spn Subset 3(a): This Subset comprises Spn genes which have homologues with GAS but do not have homologues with GBS or with any of the other published bacterial genomes at the time of the invention;

Spn Subset 4(a): This Subset comprises Spn genes which do not have homologues with either GBS or pneumococcus or with any of the other published bacterial genomes at the time of the invention.

The invention also provides polynucleotides which are conserved or specific to GBS serotypes and/or clinical isolates. Applicants have sequenced 19 GBS genes from a variety of GBS serotypes in 11 different clinical isolates. The sequences of these genes and their alignments are set forth in Tables 13-31. Polynucleotide and polypeptide sequences which are specific or conserved across one or more clinical isolates can be identified using these alignments. The following additional subsets are provided:

GBS Subset 1(b): of the 1060 GBS genes which have homologues with GAS and with pneumococcus, 47 of these GBS genes vary among the 11 clinical isolates (GBS Subset 1(b)(i)). 1013 of these GBS genes are conserved across the 11 clinical isolates (GBS Subset 1(b)(ii)). These lists can be determined by comparing the genes listed in Table 8 with the Comparative Genome Hybridization in FIG. 1.

GBS Subset 2(b): of the 225 GBS genes which have homologues with GAS, but not pneumococcus, 44 of these GBS genes vary among the 11 clinical isolates (GBS Subset 2(b)(i)). 181 of these GBS genes are conserved across the 11 clinical isolates (GBS Subset 2(b)(ii)). These lists can be determined by comparing the genes listed in Table 10 with the Comparative Genome Hybridization in FIG. 1.

GBS Subset 3(b): of the 176 GBS genes which have homologues with pneumococcus, 44 of these GBS genes vary among 11 clinical isolates (GBS Subset 3(b)(i)). 132 of these GBS genes are conserved across the 11 clinical isolates (GBS Subset 3(b)(ii)). This list can be determined by comparing the genes listed in Table 9 with the Comparative Genome Hybridization in FIG. 1.

GBS Subset 4(b): of the 683 GBS genes which do not have homologues with GAS or pneumococcus, 260 GBS genes vary among the 11 clinical isolates (GBS Subset 4(b)(i)). 423 of these GBS genes are conserved across the 11 clinical isolates (GBS Subset 4(b)(ii)). This list can be determined by comparing the genes listed in Table 11 with the Comparative Genome Hybridization in FIG. 1. GBS Subset 4(b)(ii) also includes the GBS ORF's listed on Table 12 receiving a “+” under the column “GBS specific”.

An additional 63 GBS genes have been sequenced and compared in 2-11 clinical isolates. These sequences and their alignments are provided in Tables 40-89. Polynucleotide and polypeptide sequences which are specific or conserved across one or more clinical isolates can be identified using these alignments.

The invention further provides polynucleotides which are likely recent genomic duplications in GBS. These duplications include glycosyl transferases, sortases, proteins anchored on the cell wall, 13 lactam resistance factors, and many hypothetic proteins. The GBS genes are listed in Table 4 (GBS Subset 5).

The invention is also based on the identification of a cluster of 13 adjacent genes (SAG1410-SAG1424) which is believed to encode enzymes required for synthesis of the group B carbohydrate, a coplex multiantennary structure of rhamnose, glucitol phosphate, N-acetylglucosamine, and galactose. (GBS Subset 6). Predicted proteins encoded within this cluster include seven putative glycoslytransferases, four of which are similar to rhamnosyltransferases in other streptococcal species; a putative dTDP-L-rhamnose synthase; and proteins involved in glucitol synthesis. All nine recognized GBS capsular polysaccharide types contain sialic acid residues as part of their repeating unit structure, a feature that contributes to virulence by inhibiting activation of the alternative complement pathway. See Edwards et al. (1982) J. Immunol. 128, 1278-1283.

The type V capsular polysaccharide gene cluster consists of 18 genes. (GBS Subset 6(a)). A region of glycosyltransferases and related proteins (SAG1162-SAG1170) that direct the synthesis of the type V polysaccharide repeat unit is flanked on either side by genes that are conserved in all known GBS capsule serotypes. Downstream of this region are genes that encode enzymes for the biosynthesis and activation of sialic acid (SAG1158-SAG1161). Upstream of the serotype specific region are genes (SAG1171-SAG1175) found not only in all nine GBS capsular serotypes but also in a variety of other polysaccharide-producing streptococci.

The invention is also based on the identification of GBS ORFs predicted to encode proteins carrying a signal peptide (GBS Subset 7). These GBS ORF's are listed in Table 2 receiving a “+” under the column “signal peptide”.

The invention is also based on the identification of GBS ORFs predicted to encode proteins which are anchored on the cell wall through an LPxTG motif (GBS Subset 8). These GBS ORF's are listed in Table 2 receiving a “+” under the column “sortase motif”.

The invention is also based on the identification of GBS ORFs predicted to encode lipoproteins (GBS Subset 9). These GBS ORF's are listed in Table 2 receiving a “+,” under the column “lipoprotein”.

The invention is also based on the identification of two GBS ORF's predicted to encode enzymes related to metabolism (GBS Subset 10). These GBS ORFs include a putative pullulanase (SAG1216) and a neuraminidase-related protein (SAG1932).

The invention is also based on the identification of GBS ORF's predicted to encode proteins exposed on the cell surface (GBS Subset 11). These GBS ORF's are listed in Table 2 receiving a “+” under the column “FACS”.

The invention is also based on the identification of 401 GBS ORF's from GBS strain 2603 V/R which were not detected in at least one other of the 11 tested clinical isolates (GBS Subset 12). See Comparative Hybridization Genome in FIG. 1. 364 of these 401 ORF's correspond to 15 regions containing more than 5 contiguous genes. Each region is identified in FIG. 1 by numerical yellow bullets. Each region comprises a subset as defined below:

Region 1: GBS Subset 12(a). This region is unique to GBS (SAG0218-SAG0238). This region is a possible plasmid or remnant of a phage and contains mostly hypothetical proteins.

Region 2: GBS Subset 12(b)

Region 3: GBS Subset 12(c)

Region 4: GBS Subset 12(d)

Region 5: GBS Subset 12(e)

Region 6: GBS Subset 12(f)

Region 7: GBS Subset 12(g)

Region 8: GBS Subset 12(h). This region is specific to GBS (SAG1018-SAG1037). This region comprises 20 proteins of unknown function, most of which are predicted to be membrane associated or secreted, and displays an atypical nucleotide composition.

Region 9: GBS Subset 12(i)

Region 10: GBS Subset 12(j)

Region 11: GBS Subset 12(k)

Region 12: GBS Subset 12(l)

Region 13: GBS Subset 12(m)

Region 14: GBS Subset 12(n). This region is unique to GBS and spans 33 genes (SAG1989-2021), including 25 proteins of unknown function, some of which carry a cell-wall anchor.

Region 15: GBS Subset 12(o).

This invention is also based on identification of clusters of GBS genes as set forth in FIG. 5 and Table 6. In FIG. 5, the presence of a particular gene or gene cluster is indicated in the figure by a red square and the absence of a gene or cluster by a black square. The relationship between strains based on this analysis is depicted by the tree at the top of the figure. The strains and their serotypes are indicated (NT: nontypeable). Clusters with identical profiles are reduced to a single horizontal line and the number of genes in each cluster is indicated on the right. The clusters of 5 or more genes, labeled in red text and numbered, are listed in Table 6. The 1698 genes shared by all 19 strains are labeled in green text. Applicants identified the following subsets:

GBS Subset 13 (a): Cluster 1 (from Table 6).

GBS Subset 13 (b): Cluster 2 (from Table 6).

GBS Subset 13 (c): Cluster 3 (from Table 6).

GBS Subset 13 (d): Cluster 4 (from Table 6).

GBS Subset 13 (e): Cluster 5 (from Table 6).

GBS Subset 13 (f): Cluster 6 (from Table 6).

GBS Subset 13 (g): Cluster 7 (from Table 6).

GBS Subset 13 (h): Cluster 8 (from Table 6).

GBS Subset 13 (i): Cluster 9 (from Table 6).

GBS Subset 13 (i): Cluster 10 (from Table 6).

GBS Subset 13 (k): Cluster 11 (from Table 6).

GBS Subset 13 (l): Cluster 12 (from Table 6).

GBS Subset 13 (m): Cluster 13 (from Table 6).

GBS Subset 13 (n): Cluster 14 (from Table 6).

GBS Subset 13 (o): Cluster 15 (from Table 6).

GBS Subset 13 (p): Cluster 16 (from Table 6).

GBS Subset 13 (q): 1698 ORFs shared by all strains.

The invention is also based on the identification of the polynucleotide sequences of 82 genes from up to 11 different GBS strains. 19 of these genes are listed on Table 7. A further GBS Subset 14 includes this set of polynucleotide sequences from the 11 strains and their encoded polypeptide sequences. In particular, GBS Subset 14 contains a Subset of polynucleotide fragments of 10 or more contiguous polynucleotides which are conserved between two or more strains (GBS Subset 14(a)). GBS Subset 14 further includes a Subset of polynucleotide fragments of 15 or more contiguous polynucleotides which are conserved between two or more strains (GBS Subset 14(b)). GBS Subset 14 further includes a Subset of polynucleotide fragments of 10 or more contiguous polynucleotides which are conserved between three or more strains (GBS Subset 14(c)). GBS Subset 14 further includes a Subset of polynucleotide fragments of 10 or more contiguous polynucleotides which are conserved between four or more strains (GBS Subset 14(d)).

GBS Subset 14 further includes a Subset of polypeptide fragments of 5 or more contiguous amino acids which are conserved between in two or more strains (GBS Subset 14(e)). GBS Subset 14 further includes a Subset of polypeptide fragments of 5 or more contigous amino acids which are conserved between three or more strains (GBS Subset 14(f)). GBS Subset 14 further includes a Subset of polypeptide fragments of 5 or more contiguous amino acids which are conserved between four or more strains (GBS Subset 14(g)). GBS Subset 14 further includes a Subset of polypeptide fragments of 10 or more contiguous amino acids which are conserved across two or more strains (GBS Subset 14(h)).

The invention provides for methods of screening a Streptococcal genome for a conserved or a specific genomic sequence using one or more of the Subsets of the invention.

The invention further provides for an immunogenic composition comprising a polypeptide expressed by one or more of the polynucleotides in one or more of the Subsets of the invention, and methods for designing an immunogenic composition by selecting one or more polypeptides expressed by one or more of the polynucleotides in one or more of the Subsets of the invention. Preferably, the immunogenic compositions of the invention comprise at least two, three, four or five polypeptides encoded by polynucleotides within the same Subset.

The invention further provides for methods of screening compounds for activity against a Streptococcal bacteria, which method comprises contacting the compounds with a polypeptide expressed by the polynucleotide from one of the Subsets of the invention.

The invention further provides for compositions comprising one or more of the polynucleotides, and fragments thereof, selected from the group consisting of the sequences set forth in Tables 13-31 or 40-89.

The invention further provides for compositions comprising polypeptides and fragments thereof encoded by the polynucleotides set forth in Tables 13-31 or 40-89.

The invention provides for compositions comprising polypeptides and fragments thereof set forth in Tables 13-31 or 40-89.

### BRIEF DESCRIPTION OF THE TABLES AND DRAWINGS

Table 1 comprises a complete list of GBS predicted genes, listed by SAGxxxx ORF number. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948. This table also includes the predicted amino acid size of the predicted expressed protein and the predicted function, if known.

Table 2 comprises a list of predicted and experimentally characterized surface and secreted proteins from GBS. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948.

Table 3 lists GBS genes which were shared among GBS, GAS and pneumococcus, but which were not found in any of the other completely sequenced genomes. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948.

Table 4 depicts GBS genes which are predicted to have been recently duplicated within the genome. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948.

Table 5 lists the 19 GBS strains used for comparative genome hybridisations and phylogenetic analysis.

Table 6 lists clusters of GBS genes derived from phylogenetic profiling of GBS strains based on comparative genome hybridisations. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948.

Table 7 lists the GBS genes used for phylogenetic analyses of the 19 GBS strains. The SAGxxxx ORF number corresponds to the genomic sequence for the Streptococcus agalactiae type V strain 2603 V/R available either at the TIGR website by Aug. 28, 2002 http://www.tigr.org or at the GenBank database at accession number AE009948.

Table 8 lists the 1060 GBS ORF's which are shared with GAS and pneumococcus. The ORFxxxxx reference number can be translated to SAGxxxx ORF number by using Table 32. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948.

Table 9 lists the 176 GBS ORF's which are shared with pneumococcus but which are not homologous to a GAS gene. The ORFxxxxx reference number can be translated to SAGxxxx ORF number by using Table 32. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948.

Table 10 lists the 225 GBS ORF's which are shared with GAS but which are not homologous with a pnuemococcus gene. The ORFxxxxx reference number can be translated to SAGxxxx ORF number by using Table 32. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948.

Table 11 lists 683 GBS ORF's which are not shared with either GAS or pneumococcus. The ORFxxxxx reference number can be translated to SAGxxxx ORF number by using Table 32. The SAGxxxx ORF number corresponds to the genomic sequence for the Streptococcus agalactiae type V strain 2603 V/R available either at the TIGR website by Aug. 28, 2002 at http://www.tigr.org or at the GenBank database at accession number AE009948.

Table 12 lists 315 GBS ORF's which are not shared with GAS, pneumococcus or any other published genomic sequence. The ORFxxxxx reference number can be translated to SAGxxxx ORP number by using Table 32. The SAGxxxx ORF number corresponds to the genomic sequence for the Streptococcus agalactiae type V strain 2603 V/R available either at the TIGR website by Aug. 28, 2002 at tigr.org or at the GenBank database at accession number AE009948.

Table 13 lists the polynucleotide sequences (Nos. 1301-1316 corresponding to SEQ ID NOS:1-16) of the 11 strains relating to GBS ORF SAG0466. An alignment of each of the sequences is also included.

Table 14 lists the polynucleotide sequences (Nos. 1401-1417 corresponding to SEQ NOS:17-33) of the 11 strains relating to GBS ORF SAG0471. An alignment of each of the sequences is also included.

Table 15 lists the polynucleotide sequences (Nos. 1501-1511 corresponding to SEQ NOS:34-44) of the 11 strains relating to GBS ORF SAG0492. An alignment of each of the sequences is also included.

Table 16 lists the polynucleotide sequences (Nos. 1601-1617 corresponding to SEQ ID NOS:45-61) of the 11 strains relating to GBS ORF SAG0767. An alignment of each of the sequences is also included.

Table 17 lists the polynucleotide sequences (Nos. 1701-1711 corresponding to SEQ ID NOS:62-72) of the 11 strains relating to GBS ORF SAG1086. An alignment of each of the sequences is also included.

Table 18 lists the polynucleotide sequences (Nos. 1801-1814 corresponding to SEQ ID NOS:73-86) of the 11 strains relating to GBS ORF SAG1600. An alignment of each of the sequences is also included.

Table 19 lists the polynucleotide sequences (Nos. 1901-1914 corresponding to SEQ ID NOS:87-100) of the 11 strains relating to GBS ORF SAG1680. An alignment of each of the sequences is also included.

Table 20 lists the polynucleotide sequences (Nos. 2001-2010 corresponding to SEQ ID NOS: 101-110) of the 11 strains relating to GBS ORF SAG1723. An alignment of each of the sequences is also included.

Table 21 lists the polynucleotide and polypeptide sequences (Nos. 2101-2124 corresponding to SEQ ID NOS:111-134) of the 11 strains relating to GBS ORF SAG0079. An alignment of each of the sequences is also included.

Table 22 lists the polynucleotide and polypeptide sequences (Nos. 2201-2222 corresponding to SEQ ID NOS:135-156) of the 11 strains relating to GBS ORF SAG0093. An alignment of each of the sequences is also included.

Table 23 lists the polynucleotide and polypeptide sequences (Nos. 2301-2323 corresponding to SEQ ID NOS:157-179) of the 11 strains relating to GBS ORF SA00163. An alignment of each of the sequences is also included.

Table 24 lists the polynucleotide and polypeptide sequences (Nos. 2401-2422 corresponding to SEQ ID NOS:180-201) of the 11 strains relating to GBS ORF SAG0290. An alignment of each of the sequences is also included.

Table 25 lists the polynucleotide and polypeptide sequences (Nos. 2501-2521 corresponding to SEQ ID NOS:202-222) of the 11 strains relating to GBS ORF SAG0368. An alignment of each of the sequences is also included.

Table 26 lists the polynucleotide and polypeptide sequences (Nos. 2601-2618 corresponding to SEQ ID NOS:223-240) of the 11 strains relating to GBS ORF SAG0503. An alignment of each of the sequences is also included.

Table 27 lists the polynucleotide and polypeptide sequences (Nos. 2701-2722 corresponding to SEQ ID NOS:241-262) of the 11 strains relating to GBS ORF 5AG1473. An alignment of each of the sequences is also included.

Table 28 lists the polynucleotide and polypeptide sequences (Nos. 2801-2822 corresponding to SEQ ID NOS:263-284) of the 11 strains relating to GBS ORF SAG1552. An alignment of each of the sequences is also included.

Table 29 lists the polynucleotide and polypeptide sequences (Nos. 2901-2922 corresponding to SEQ ID NOS:285-306) of the 11 strains relating to GBS ORF SAG1641. An alignment of each of the sequences is also included.

Table 30 lists the polynucleotide and polypeptide sequences (Nos. 3001-3020 corresponding to SEQ ID NOS:307-326) of the 11 strains relating to GBS ORF SAG2147. An alignment of each of the sequences is also included.

Table 31 lists the polynucleotide and polypeptide sequences (Nos. 3101-3122 corresponding to SEQ ID NOS:327-348) of the 11 strains relating to GBS ORF SAG2148. An alignment of each of the sequences is also included.

Table 32 provides a conversion table for the ORFxxxx reference numbers to the SAGxxxx reference numbers. The SAGxxxx ORF number corresponds to the genomic sequence for the Streptococcus agalactia type V strain 2603 V/R available either at the TIGR website by Aug. 28, 2002 at http://www.tigr.org or at the GenBank database at accession number AE009948.

Table 33 lists the 1006 GAS ORF's which are shared with GBS and Spn. The sequences corresponding to these ORFs were published in GenBank, Accession No. AAK33146 (protein sequence). A link to the corresponding polynucleotide sequence is also available. The numbers for the GAS ORF refer directly to their GenBank entries.

Table 34 lists the 212 GAS ORF's which are shared with GBS but which do not have homologues with pneumococcus. The sequences corresponding to these ORFs were published in GenBank, Accession No AAK33146 (protein sequence). A link to the corresponding polynucleotide sequence is also available. The numbers for the GAS ORF refer directly to their GenBank entries.

Table 35 lists the 62 GAS ORF's which have homologues with pneumococcus but which do not have homologues with GBS. The sequences corresponding to these ORFs were published in GenBank, Accession No. AAK3146 (protein sequence). A link to the corresponding polynucleotide sequence is also available. The numbers for the GAS ORF refer directly to their GenBank entries.

Table 36 lists the 1034 Spn ORFs which are shared with GBS and GAS. These ORF's were published in GenBank. The numbers for Spn correspond to the entry for AE005672.

Table 37 lists the 195 Spn ORF's which are shared with GBS but do not have homologues with GAS. These ORF's were published in GenBank. The numbers for Spn correspond to the entry for AE005672.

Table 38 lists the 74 Spn ORFS which are shared with GAS but do not have homologues with GBS. These ORFs were published in GenBank. The numbers for Spn correspond to the entry for AE005672.

Table 40 lists the polynucleotide and polypeptide sequences (Nos. 4001-4018 corresponding to SEQ ID NOS:349-366) of 8 strains relating to GBS ORF SAG0635. An alignment of the polynucleotide and polypeptide sequences is also included.

Table 41 lists the polynucleotide and polypeptide sequences (Nos. 4101-4118 corresponding to SEQ ID NOS:367-384) of 8 strains relating to GBS ORF SAG0649. An alignment of the polynucleotide and polypeptide sequences is also included.

Table 42 lists the polynucleotide and polypeptide sequences (Nos. 4201-4222 corresponding to SEQ ID NOS:385-406) of 10 strains relating to GBS ORF SAG0764. An alignment of the polynucleotide and polypeptide sequences is also included.

Table 43 lists the polynucleotide and polypeptide sequences (Nos. 4301-4323 corresponding to SEQ ID NOS:407-429) of 10 strains relating to GBS ORF SAG0079. An alignment of the polynucleotide and polypeptide sequences is also included.

Table 44 lists the polynucleotide and polypeptide sequences (Nos. 4401-4422 corresponding to SEQ ID NOS:430-451) of 10 strains relating to GBS ORF SAG0416. An alignment of the polynucleotide and polypeptide sequences is also included.

Table 45 lists the polynucleotide and polypeptide sequences (Nos. 4501-4511 corresponding to SEQ ID NOS:452-462) of 5 strains relating to GBS ORF SAG1404. An alignment of the polynucleotide and polypeptide sequences is also included.

Table 46 lists the polynucleotide and polypeptide sequences (Nos. 4601-4623 corresponding to SEQ ID NOS:463-485) of 10 strains relating to GBS ORF SAG1615. An alignment of the polynucleotide and polypeptide sequences is also included.

Table 47 lists the polynucleotide and polypeptide sequences (Nos. 4701-4722 corresponding to SEQ ID NOS:486-507) of 10 strains relating to GBS ORF SAG0739. An alignment of the polynucleotide and polypeptide sequences is also included.

Table 48 lists the polynucleotide and polypeptide sequences (Nos. 4801-4824 corresponding to SEQ ID NOS:508-531) of 10 strains relating to GBS ORF SAG1474. An alignment of the polynucleotide and polypeptide sequences is also included.

Table 49 lists the polynucleotide and polypeptide sequences (Nos. 4901-4922 corresponding to SEQ ID NOS:532-553) of 10 strains relating to GBS ORF SAG1502. An alignment of the polynucleotide and polypeptide sequences is also included.

Table 50 lists the polynucleotide and polypeptide sequences (Nos. 5001-5006 corresponding to SEQ ID NOS:554-559) of 2 strains relating to GBS ORF SAG1024. An alignment of the polynucleotide and polypeptide sequences is also included.

Table 51 lists the polynucleotide and polypeptide sequences (Nos. 5101-5117 corresponding to SEQ ID NOS:560-576) of 7 strains relating to GBS ORF SAG0677. An alignment of the polynucleotide and polypeptide sequences is also included.

Table 52 lists the polynucleotide and polypeptide sequences (Nos. 5201-5223 corresponding to SEQ ID NOS:577-599) of 10 strains relating to GBS ORF SAG1823. An alignment of the polynucleotide and polypeptide sequences is also included.

Table 53 lists the polynucleotide and polypeptide sequences (Nos. 5301-5322 corresponding to SEQ ID NOS:600-621) of 10 strains relating to GBS ORF SAG0755. An alignment of the polynucleotide and polypeptide sequences is also included.

Table 54 lists the polynucleotide and polypeptide sequences (Nos. 5401-5422 corresponding to SEQ ID NOS:622-643) of 10 strains relating to GBS ORF SAG0949. An alignment of the polynucleotide and polypeptide sequences is also included.

Table 55 lists the polynucleotide and polypeptide sequences (Nos. 5501-5522 corresponding to SEQ ID NOS:644-665) of 10 strains relating to GBS ORF SAG1592. An alignment of the polynucleotide and polypeptide sequences is also included.

Table 56 lists the polynucleotide and polypeptide sequences (Nos. 5601-5622 corresponding to SEQ ID NOS:666-687) of 10 strains relating to GBS ORF SAG0806. An alignment of the polynucleotide and polypeptide sequences is also included.

Table 57 lists the polynucleotide and polypeptide sequences (Nos. 5701-5722 corresponding to SEQ ID NOS:688-709) of 10 strains relating to GBS ORF SAG1488. An alignment of the polynucleotide and polypeptide sequences is also included.

Table 58 lists the polynucleotide and polypeptide sequences (Nos. 5801-5821 corresponding to SEQ ID NOS:710-730) of 10 strains relating to GBS ORF SAG0182. An alignment of the polynucleotide and polypeptide sequences is also included.

Table 59 lists the polynucleotide and polypeptide sequences (Nos. 5901-5923 corresponding to SEQ ID NOS:731-753) of 10 strains relating to GBS ORF SAG2147. An alignment of the polynucleotide and polypeptide sequences is also included.

Table 60 lists the polynucleotide and polypeptide sequences (Nos. 6001-6022 corresponding to SEQ ID NOS:754-775) of 10 strains relating to GBS ORF SAG1945. An alignment of the polynucleotide and polypeptide sequences is also included.

Table 61 lists the polynucleotide and polypeptide sequences (Nos. 6101-6106 corresponding to SEQ ID NOS:776-781) of 2 strains relating to GBS ORF SAG1030. An alignment of the polynucleotide and polypeptide sequences is also included.

Table 62 lists the polynucleotide and polypeptide sequences (Nos. 6201-6222 corresponding to SEQ ID NOS:782-803) of 10 strains relating to GBS ORF SAG0690. An alignment of the polynucleotide and polypeptide sequences is also included.

Table 63 lists the polynucleotide and polypeptide sequences (Nos. 6301-6321 corresponding to SEQ ID NOS:804-824) of 10 strains relating to GBS ORF SAG1912. An alignment of the polynucleotide and polypeptide sequences is also included.

Table 64 lists the polynucleotide and polypeptide sequences (Nos. 6401-6423 corresponding to SEQ ID NOS:825-847) of 10 strains relating to GBS ORE SAG0827. An alignment of the polynucleotide and polypeptide sequences is also included.

Table 65 lists the polynucleotide and polypeptide sequences (Nos. 6501-6518 corresponding to SEQ ID NOS:848-865) of 8 strains relating to GBS ORE SAG0231. An alignment of the polynucleotide and polypeptide sequences is also included.

Table 66 lists the polynucleotide and polypeptide sequences (Nos. 6601-6622 corresponding to SEQ ID NOS:866-887) of 10 strains relating to GBS ORF SAG0754. An alignment of the polynucleotide and polypeptide sequences is also included.

Table 67 lists the polynucleotide and polypeptide sequences (Nos. 6701-6721 corresponding to SEQ ID NOS:888-908) of 10 strains relating to GBS ORE SAG0475. An alignment of the polynucleotide and polypeptide sequences is also included.

Table 68 lists the polynucleotide and polypeptide sequences (Nos. 6801-6822 corresponding to SEQ ID NOS:909-930) of 10 strains relating to GBS ORE SAG0499. An alignment of the polynucleotide and polypeptide sequences is also included.

Table 69 lists the polynucleotide and polypeptide sequences (Nos. 6901-6922 corresponding to SEQ ID NOS:931-952) of 10 strains relating to GBS ORF SAG0032. An alignment of the polynucleotide and polypeptide sequences is also included.

Table 70 lists the polynucleotide and polypeptide sequences (Nos. 7001-7006 corresponding to SEQ ID NOS:953-958) of 2 strains relating to GBS ORF SAG1280. An alignment of the polynucleotide and polypeptide sequences is also included.

Table 71 lists the polynucleotide and polypeptide sequences (Nos. 7101-7122 corresponding to SEQ ID NOS:959-980) of 10 strains relating to GBS ORE SAG1333. An alignment of the polynucleotide and polypeptide sequences is also included.

Table 72 lists the polynucleotide and polypeptide sequences (Nos. 7201-7222 corresponding to SEQ ID NOS:981-1002) of 10 strains relating to GBS ORE SAG0941. An alignment of the polynucleotide and polypeptide sequences is also included.

Table 73 lists the polynucleotide and polypeptide sequences (Nos. 7301-7320 corresponding to SEQ ID NOS:1003-1022) of 10 strains relating to GBS ORF SAG0981. An alignment of the polynucleotide and polypeptide sequences is also included.

Table 74 lists the polynucleotide and polypeptide sequences (Nos. 7401-7422 corresponding to SEQ ID NOS:1023-1044) of 10 strains relating to GBS ORF SAG1572. An alignment of the polynucleotide and polypeptide sequences is also included.

Table 75 lists the polynucleotide and polypeptide sequences (Nos. 7501-7522 corresponding to SEQ ID NOS:1045-1066) of 10 strains relating to GBS ORF SAG0671. An alignment of the polynucleotide and polypeptide sequences is also included.

Table 76 lists the polynucleotide and polypeptide sequences (Nos. 7601-7622 corresponding to SEQ ID NOS:1067-1088) of 10 strains relating to GBS ORF SAG0260. An alignment of the polynucleotide and polypeptide sequences is also included.

Table 77 lists the polynucleotide and polypeptide sequences (Nos. 7701-7722 corresponding to SEQ ID NOS:1089-1110) of 10 strains relating to GBS ORF SAG2059. An alignment of the polynucleotide and polypeptide sequences is also included.

Table 78 lists the polynucleotide and polypeptide sequences (Nos. 7801-7822 corresponding to SEP ID NOS:1111-1132) of 10 strains relating to GBS ORF SAG1016. An alignment of the polynucleotide and polypeptide sequences is also included.

Table 79 lists the polynucleotide and polypeptide sequences (Nos. 7901-7922 corresponding to SEQ ID NOS:1133-1154) of 10 strains relating to GBS ORF SAG2150. An alignment of the polynucleotide and polypeptide sequences is also included.

Table 80 lists the polynucleotide and polypeptide sequences (Nos. 8001-8006 corresponding to SEQ ID NOS:1155-1160) of 2 strains relating to GBS ORF SAG1266. An alignment of the polynucleotide and polypeptide sequences is also included.

Table 81 lists the polynucleotide and polypeptide sequences (Nos. 8101-8122 corresponding to SEQ ID NOS:1161-1182) of 10 strains relating to GBS ORF SAG0011. An alignment of the polynucleotide and polypeptide sequences is also included.

Table 82 lists the polynucleotide and polypeptide sequences (Nos. 8201-8222 corresponding to SEQ ID NOS:1183-1204) of 10 strains relating to GBS ORF SAG0165. An alignment of the polynucleotide and polypeptide sequences is also included.

Table 83 lists the polynucleotide and polypeptide sequences (Nos. 8301-8322 corresponding to SEQ ID NOS: 1205-1226) of 10 strains relating to GBS ORF SAG0108. An alignment of the polynucleotide and polypeptide sequences is also included.

Table 84 lists the polynucleotide and polypeptide sequences (Nos. 8401-8422 corresponding to SEQ ID NOS:1227-1248) of 10 strains relating to GBS ORF SAG0267. An alignment of the polynucleotide and polypeptide sequences is also included.

Table 85 lists the polynucleotide and polypeptide sequences (Nos. 8501-8522 corresponding to SEQ ID NOS:1249-1270) of 10 strains relating to GBS ORF SAG1361. An alignment of the polynucleotide and polypeptide sequences is also included.

Table 86 lists the polynucleotide and polypeptide sequences (Nos. 8601-8622 corresponding to SEQ ID NOS:1271-1292) of 10 strains relating to GBS ORF SAG1393. An alignment of the polynucleotide and polypeptide sequences is also included.

Table 87 lists the polynucleotide and polypeptide sequences (Nos. 8701-8718 corresponding to SEQ ID NOS:1293-1310) of 8 strains relating to GBS ORF SAG0645. An alignment of the polynucleotide and polypeptide sequences is also included.

Table 88 lists the polynucleotide and polypeptide sequences (Nos. 8801-8822 corresponding to SEQ ID NOS:1311-1332) of 10 strains relating to GBS ORF SAG0477. An alignment of the polynucleotide and polypeptide sequences is also included.

Table 89 lists the polynucleotide and polypeptide sequences (Nos. 8901-8922 corresponding to SEQ ID NOS:1333-1354) of 10 strains relating to GBS ORF SAG1350. An alignment of the polynucleotide and polypeptide sequences is also included.

FIG. 1 is a circular representation of the GBS genome and comparative hybridisations using microarrays. A color version of FIG. 1 can be found in Tettelin et al., PNAS (2002) 99(19): 12391-12396 and online at the Proc. Natl. Acad. Sci. USA website.

FIG. 2 is a schematic representation of in silico comparisons between streptococci. A color version of FIG. 2 can be found in Tettelin et al., PNAS (2002) 99(19): 12391-12396 and online at the Proc. Natl. Acad. Sci. USA website.

FIG. 3 depicts a phylogenetic tree of GBS strains based on PCR sequences.

FIG. 4 depicts a linear representation of the GBS genome. A color version of FIG. 4 can be found in the supporting information to Tettelin et al., PNAS (2002) 99(19): 12391-12396 available online at the Proc. Natl. Acad. Sci. USA website.

FIG. 5 demonstrates phylogenetic profiling of GBS strains based on comparative genome hybridisations. A color version of FIG. 5 can be found in the supporting information to Tettelin et al., PNAS (2002) 99(19):12391-12396 available online at the Proc. Natl. Acad. Sci. USA website.

### DETAILED DESCRIPTION OF THE INVENTION

The invention relates to polynucleotides which are conserved or specific to one or more species of Streptococcus, Streptococcus species serotypes, and/or serotype isolates. In particular, the invention relates to polynucleotides from Streptococcus which are conserved or specific to one or more of the species of S. pneumoniae (“pneumococcus” or “S. pn.”), S. pyogenes (“group A streptococcus” or “GAS”), and S. agalactiae (“group B streptococcus” or “GBS”). The invention further relates to polynucleotides which are conserved or specific to one or more Streptococcal species serotypes, such as GBS serotypes Ia, Ib, II, III, IV, V, VI, VII, and VIII. The invention still further relates to polynucleotides which are conserved or specific to one or more clinical isolates of a Streptococcus species.

In order to facilitate an understanding of the invention, selected terms used in the application will be discussed below.

As used herein, the phrase “species of Streptococcus” generally refers to species of the Streptoccus family, including S. pneumoniae (“pneumococcus” or “S.pn.”), S. pyogenes (“group A streptococcus” or “GAS”) and S. agalactiae (“group B streptococcus” or “GBS”).

As used herein, the phrase “Streptococcus species serotypes” generally refers to subdivisions based on a distinguishing characteristic within a specific Streptococcus species. The distinguishing characteristic can be identified by any of a wide range of diagnostic tools.

For instance, GBS is generally recognized as comprising at least nine subdividing serotypes based on the structure of their polysaccharide capsule.

As used herein, the phrases “serotype isolates” or “clinical isolates” generally refer to specific isolated bacterial strains of a specific Streptococcal species and serotype.

As used herein in reference to bacterial genomes, the phrases “conserved” or “shared” generally refer to genomic sequences which have homologues in the two or more genomes in the reference. Homology references, as used in this application, are generally based on comparisons using FASTA3. See Pearson (2000) Methods Mol. Biol. 132 185-219. When the homology reference involves a comparison between genes in GBS, GAS or Spn, homologous or shared genes are typically defined by using a FASTA3 P value cutoff of 10⁻¹⁵. Where the homology reference involves a comparison between GBS, GAS or Spn and all other completely sequenced genomes, homologous or shared genes are typically defined by using a FASTA3 P value cutoff of 10⁻⁵ or lower.

As used herein in reference to bacterial genomes, the phrases “specific to” or “not shared” generally refer to genomic sequences which do not have homologues in the two or more genomes in the reference.

Other software programs to compare identity and to determine homology between nucleotide sequences are known in the art, for example those described in section 7.7.18 of Current Protocols in Molecular Biology (F. M. Ausubel et al., eds., 1987) Supplement 30. A preferred alignment program is GCG Gap (Genetics Computer Group, Wisconsin, Suite Version 10.1), preferably using default parameters, which are as follows: open gap=3; extend gap=1.

Sequences within a Subset of the invention include sequences which hybridize to the listed genes. Hybridization reactions can be performed under conditions of different “stringency”. Conditions that increase stringency of a hybridization reaction of widely known and published in the art [e.g. page 7.52 of Sambrook et al. (1989) Molecular Cloning: A Laboratory Manual. NY, Cold Spring Harbor Laboratory]. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see Sambrook et al.; RNA Methodologies (Farrell, 1998) (Academic Press; ISBN 0-12-249695-7); Current Protocols in Molecular Biology (F. M. Ausubel et al., eds., 1987) Supplement 30; Short protocols in molecular biology (4th edition, 1999) Ausubel et al. eds. ISBN 0-471-32938-X; U.S. Pat. No. 5,707,829 etc.].

Identity between polypeptide sequences can be determined using software programs known in the art, for example those described in section 7.7.18 of Current Protocols in Molecular Biology (F. M. Ausubel et al., eds., 1987) Supplement 30. A preferred alignment is determined by the Smith-Waterman homology search algorithm [Smith & Waterman (1981) Adv. Appl. Math. 2: 482-489.] using an affine gap search with a gap open penalty of 12 and a gap extension penalty of 2, BLOSUM matrix 62.

Typically, 50% identity or more between two proteins may be considered to be an indication of functional equivalence. References to a percentage sequence identity between two amino acid sequences means that, when aligned, that percentage of amino acids are the same in comparing the two sequences.

The terms “polypeptide”, “protein” and “amino acid sequence” as used herein generally refer to a polymer of amino acid residues and are not limited to a minimum length of the product. Thus, peptides, oligopeptides, dimers, multimers, and the like, are included within the definition. Both full-length proteins and fragments thereof are encompassed by the definition. Minimum fragments of polypeptides useful in the invention can be at least 3, 4, 5, 6, 7, 8, 9, 10, 11, 12, 13, 14, 15, 18, 20, 25, 30, 35, 40 or 50 amino acids. Typically, polypeptides useful in this invention can have a maximum length suitable for the intended application. Generally, the maximum length is not critical and can easily be selected by one skilled in the art.

Reference to polypeptides and the like also includes derivatives of the amino acid sequences of the invention. Such derivatives can include postexpression modifications of the polypeptide, for example, glycosylation, acetylation, phosphorylation, and the like. Amino acid derivatives can also include modifications to the native sequence, such as deletions, additions and substitutions (generally conservative in nature), so long as the protein maintains the desired activity. These modifications may be deliberate, as through site-directed mutagenesis, or may be accidental, such as through mutations of hosts which produce the proteins or errors due to PCR amplification. Furthermore, modifications may be made that have one or more of the following effects: reducing toxicity; facilitating cell processing (e.g., secretion, antigen presentation, etc.); and facilitating presentation to B-cells and/or T-cells.

A “recombinant” protein is a protein which has been prepared by recombinant DNA techniques as described herein. In general, the gene of interest is cloned and then expressed in transformed organisms, as described further below. The host organism expressed the foreign gene to produce the protein under expression conditions. The polypeptides of the invention may be prepared by recombinant means.

The term “polynucleotide”, as known in the art, generally refers to a nucleic acid molecule. A “polynucleotide” can include both double- and single-stranded sequences and refers to, but is not limited to, cDNA from viral, prokaryotic or eukaryotic mRNA, genomic RNA and DNA sequences from viral (e.g. RNA and DNA viruses and retroviruses) or prokaryotic DNA, and especially synthetic DNA sequences. The term also captures sequences that include any of the known base analogs of DNA and RNA, and includes modifications such as deletions, additions and substitutions (generally conservative in nature), to the native sequence, so long as the nucleic acid molecule encodes a therapeutic or antigenic protein. These modifications may be deliberate, as through site-directed mutagenesis, or may be accidental, such as through mutations of hosts that produce the antigens. Modifications of polynucleotides may have any number of effects including, for example, facilitating expression of the polypeptide product in a host cell.

The term “polynucleotide” further includes DNA, RNA, DNA/RNA hybrids, DNA and RNA analogues such as those containing modified backbones (with modifications in the sugar and/or phosphates e.g. phosphorothioates, phosphoramidites etc.), and also peptide nucleic acids (PNA) and any other polymer comprising purine and pyrimidine bases or other natural, chemically or biochemically modified, non-natural, or derivatized nucleotide bases etc. Nucleic acid according to the invention can be prepared in many ways (e.g. by chemical synthesis, from genomic or cDNA libraries, from the organism itself etc.) and can take various forms (e.g. single stranded, double stranded, vectors, probes etc.).

A polynucleotide can encode a biologically active (e.g., immunogenic or therapeutic) protein or polypeptide. Depending on the nature of the polypeptide encoded by the polynucleotide, a polynucleotide can include as little as 10 nucleotides, e.g., where the polynucleotide encodes an antigen. The polynucleotides of the invention may comprise at least 10, 13, 15, 18, 20, 22, 25, 30, 35, 40, 45, 50, 55, 60, 65, 70, 80, 90 or 100 consecutive polynucleotides.

By “isolated” is meant, when referring to a polynucleotide or a polypeptide, that the indicated molecule is separate and discrete from the whole organism with which the molecule is found in nature or, when the polynucleotide or polypeptide is not found in nature, is sufficiently free of other biological macromolecules so that the polynucleotide or polypeptide can be used for its intended purpose.

“Antibody” as known in the art includes one or more biological moieties that, through chemical or physical means, can bind to or associate with an epitope of a polypeptide of interest. The antibodies of the invention specifically bind to infectious prion conformations. The term “antibody” includes antibodies obtained from both polyclonal and monoclonal preparations, as well as the following: hybrid (chimeric) antibody molecules (see, for example, Winter et al. (1991) Nature 349: 293-299; and U.S. Pat. No. 4,816,567; F(ab′)2 and F(ab) fragments; F, molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5897-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain immunological binding properties of the parent antibody molecule. The term “antibody” further includes antibodies obtained through non-conventional processes, such as phage display.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. See, e.g., Cote, et al. Monoclonal Antibodies and Cancer Therapy, Alan R. Liss, 1985, p 77.

An “immunogenic composition” as used herein refers to a composition that comprises an antigenic molecule where administration of the composition to a subject results in the development in the subject of a humoral and/or a cellular immune response to the antigenic molecule of interest. The immunogenicity of the composition or the antigenicity of the molecule may be facilitated by the use of an adjuvant.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B. N. Raven Press, New York, N.Y.

It is understood that the antibodies and methods of this invention are not limited to particular formulations or process parameters as such may, of course, vary. It is also to be understood that the terminology used herein is for the purpose of describing particular embodiments of the invention only, and is not intended to be limiting.

All publications, patents and patent applications cited herein are hereby incorporated by reference in their entirety.

### Vaccines and Immunisation

The invention provides an immunogenic composition comprising a polypeptide, or a fragment thereof, which is encoded by a polynucleotide sequence which is conserved across one or more species of Streptococcus.

The polynucleotide is preferably conserved across one or more species of Streptococcus selected from the group consisting of GBS, GAS and pneumococcus. In one embodiment, the polynucleotide is a GBS polynucleotide which is homologous with at least one gene from both GAS and pneumococcus. Preferably, the GBS polynucleotide is selected from GBS Subset 1, which includes 1060 GBS genes which have homologues with both GAS and pneumococcus (Table 8).

In another embodiment, the polynucleotide is a GAS polynucleotide which is homologous with at least one gene from both GBS and pneumococcus. Preferably, the GAS polynucleotide is selected from GAS Subset 1, which includes 1006 GAS genes which have homologues with both GBS and pneumococcus.

In another embodiment, the polynucleotide is a pneumococcal polynucleotide which is homologous with at least one gene both GAS and GBS. Preferably, the pneumococcus polynucleotide is selected from Spn Subset 1, which includes 1034 pneumococcal genes which have homologous with both GBS and GAS.

In another embodiment, the polynucleotide is a GBS polynucleotide which is homologous with at least one gene from GAS. Preferably, the polynucleotide is selected from one of the genes listed GBS Subset 2, which includes 225 GBS genes which have homologues with GAS, but not with pneumococcus.

In another embodiment, the polynucleotide is a GBS polynucleotide which is homologous with at least one gene from pneumococcus. Preferably, the polynucleotide is selected from GBS Subset 3, which includes 176 GBS genes which have homologues with pneumococcus.

In another embodiment, the polynucleotide is a GAS polynucleotide which is homologous with at least one gene from GBS. Preferably, the polynucleotide is selected from GAS Subset 2, which includes 212 GAS genes which have a homologue with GBS.

In another embodiment, the polynucleotide is a GAS polynucleotide which is homologous with at least one gene from pneumoccus. Preferably, the polynucleotide is selected from GAS Subset 3, which includes 62 GAS genes which have a homologue with pneumococcus.

In another embodiment, the polynucleotide is a pneumococcus polynucleotide which is homologous with at least one gene from GBS. Preferably, the polynucleotide is selected from Spn Subset 2, which includes 195 Spn genes which have a homologue with GBS.

In another embodiment, the polynucleotide is a pneumococcus polynucleotide which is homologous with at least one gene from GAS. Preferably, the polynucleotide is selected from Spn Subset 3, which includes 74 Spn genes which have a homologue with GAS.

The invention further provides an immunogenic composition comprising a polypeptide, or a fragment thereof, which is encoded by a polynucleotide sequence which is specific to one or more species of Streptococcus.

The invention further provides an immunogenic composition comprising a polypeptide, or a fragment thereof, which is encoded by a polynucleotide which is specific to GBS, GAS and pneumococcus. In one embodiment, the polynucleotide is a GBS polynucleotide which is homologous to at least one gene from both GAS and pneumococcus. Preferably, the GBS polynucleotide is selected from GBS Subset 1. In an alternative embodiment, the polynucleotide is a GBS polynucleotide which is homologous to at least one gene from both GAS and pneumococcus, but which is not homologous to a gene in any other published bacterial genome at the time of the invention. Preferably, the GBS polynucleotide is selected from one of the 12 GBS genes included in GBS Subset 1(a). (Table 3).

In another embodiment, the polynucleotide is a GAS polynucleotide which is homologous to at least one gene in both GBS and pneumococcus. Preferably, the GAS polynucleotide is selected from GAS Subset 1. In another embodiment, the polynucleotide is a GAS polynucleotide which is homologous to at least one gene in both GBS and pneumococcus but which is not homologous to any gene in any other published bacterial genome at the time of the invention. Preferably, the GAS polynucleotide is selected from GAS Subset 1(a).

Alternatively, the polynucleotide is a pneumoccus polynucleotide which is homologous to at least one gene in both GBS and GAS. Preferably, the pneumococcus polynucleotide is selected from Spn Subset 1(a). In another embodiment, the polynucleotide is a pneumoccus polynucleotide which is homologous to at least one gene in both GBS and GAS but which does not have a homologue in any other published bacterial genome at the time of the invention. Preferably, the pneumococcus polynucleotide is selected from Spn Subset 1(a).

The invention further provides an immunogenic composition comprising a polypeptide, or a fragment thereof, which is encoded by a polynucleotide sequence which is specific to GBS. In one embodiment, the polynucleotide is a GBS polynucleotide which is not homologue to a gene in either GAS or pneumococcus. Preferably, the GBS polynucleotide is selected from one of the 683 GBS genes included in GBS Subset 4. In a further embodiment, the polynucleotide is a GBS polynucleotide which is not homologous to a gene in either GAS or pneumococcus or any other published bacterial genome at the time of the invention. Preferably, the GBS polynucleotide is selected from one of the 315 GBS genes in GBS Subset 4(a).

The invention further provides an immunogenic composition comprising a polypeptide, or a fragment thereof, which is encoded by a polynucleotide sequence which is specific to GAS. In one embodiment, the polynucleotide is a GAS polynucleotide which is not homologous to a gene in either GBS or pneumococcus. Preferably, the GBS polynucleotide is selected from one of the 416 GAS genes included in GAS Subset 4. In a further embodiment, the polynucleotide is a GAS polynucleotide which does not have a homologue in either GBS or pneumococcus or in any other published bacterial genome at the time of the invention. Preferably, the GAS polynucleotide is selected from GAS Subset 4(a).

The invention further provides an immunogenic composition comprising a polypeptide, or a fragment thereof, which is encoded by a polynucleotide sequence which is specific to pneumococcus. In one embodiment, the polynucleotide is a pneumococcus polynucleotide which is not homologous to a gene in either GBS or GAS. Preferably, the pneumococcus polynucleotide is selected from one of the 836 Spn genes included in Spn Subset 4. In a further embodiment, the polynucleotide is a pneumococcus polynucleotide which does not have a homologue in either GBS or GAS or in any other published bacterial genome at the time of the invention. Preferably, the pneumococcus polynucleotide is selected from Spn Subset 4(a).

The invention further provides an immunogenic composition comprising a polypeptide, or a fragment thereof, which is encoded by a polynucleotide sequence which is specific to GBS and GAS. In one embodiment, the polynucleotide is a GBS polynucleotide which is homologous to at least one gene from GAS but is not homologous to a gene from pneumococcus. Preferably, the GBS polynucleotide is selected from one of the 225 GBS genes included in GBS Subset 2. In another embodiment, the GBS polynucleotide is homologous to at least one gene from GAS but is not homologous to any gene from pneumococcus and does not have a homologue in any other published bacterial genome at the time of the invention. Preferably, the GBS polynucleotide is selected from GBS Subset 2(a).

In another embodiment, the polynucleotide is a GAS polynucleotide which is homologous to at least one gene from GBS but is not homologous to any gene from pneumococcus. Preferably, the GAS polynucleotide is selected from one of the 212 GAS genes included in GAS Subset 2. In another embodiment, the GAS polynucleotide is homologous to at least one gene from GBS but is not homologous to any gene from pneumococcus and does not have a homologous gene with any other published bacterial genome at the time of the invention. Preferably, the GAS polynucleotide is a selected from GAS Subset 2(a).

The invention further provides an immunogenic composition comprising a polypeptide, or a fragment thereof, which is encoded by a polynucleotide sequence which is specific to GBS and pneumococcus. In one embodiment, the polynucleotide is a GBS polynucleotide which is homologous to at least one gene from pneumococcus but is not homologous to any gene from GAS. Preferably, the GBS polynucleotide is selected from one of the 176 GBS genes included in GBS Subset 3. In another embodiment, the polynucleotide is a GBS polynucleotide which is homologous with at least one gene from pneumococcus but is not homologous with any GAS polynucleotide and does not have a homologous gene in any of the other published bacterial genomes at the time of the invention. Preferably, the GBS polynucleotide is selected from GBS

Subset 3(a).

In another embodiment, the polynucleotide is a pneumococcus polynucleotide which is homologous with at least one gene from GBS, but is not homologous with any gene from GAS. Preferably, the pneumococcus polynucleotide is selected from one of the 195 Spn genes included in Spn Subset 2. In another embodiment, the polynucleotide is a pneumococcus polynucleotide which is homologous with at least one gene from GBS, but is not homologous with any gene from GAS and does not have a homologous gene in any other published bacterial genome at the time of the invention. Preferably, the pneumococcus polynucleotide is selected from Spn Subset 3(a).

The invention further provides an immunogenic composition comprising a polypeptide, or a fragment thereof which is encoded by a polynucleotide sequence which is specific to GAS and pneumococcus. In one embodiment, the polynucleotide is a GAS polynucleotide which is homologous with at least one gene from pneumococcus but is not homologous with any gene from GBS. Preferably, the GAS polynucleotide is selected from one of the 62 GAS genes included in GAS Subset 3. In another embodiment, the polynucleotide is a GAS polynucleotide which is homologous with at least one gene from pneumococcus but is not homologous with any gene from GBS and is not homologous with any gene of any published bacterial genome at the time of the invention. Preferably, the GAS polynucleotide is selected from GAS Subset 3(a).

In another embodiment, the polynucleotide is a pneumococcus polynucleotide which is homologous with at least one GAS polynucleotide, but is not homologous with any GBS gene. Preferably, the pneumococcus polynucleotide is selected from one of the 74 Spn genes included in Spn Subset 3. In another embodiment, the polynucleotide is a pneumococcus polynucleotide which is homologous with at least one gene from GAS, but is not homologous with any gene from GBS or with a gene from any other published bacterial genome at the time of the invention. Preferably, the pneumococcus polynucleotide is selected from Spn Subset 3(a).

The invention further provides an immunogenic composition comprising a polypeptide, or a fragment thereof, which is encoded by a polynucleotide sequence which is specific to one or more Streptococcal species serotypes. Preferably, the polynucleotide is specific to a Streptococcal species serotype selected from the Streptococcal species GBS, GAS and pneumococcus. More preferably, the polynucleotide is specific to one or more GBS serotypes selected from the group consisting of GBS serotype Ia, Ib, II, III, IV, V, VI, VII and VIII.

The invention further provides an immunogenic composition comprising a polypeptide, or a fragment thereof, which is encoded by a polynucleotide sequence which is conserved across one or more Streptococcal species serotypes. Preferably, the polynucleotide is specific to a Streptococcal species serotype selected from the Streptococcal species GBS, GAS and pneumococcus. More preferable, the polynucleotide is conserved across one or more GBS serotypes selected from the group consisting of GBS serotype Ia, Ib, II, I, IV, V, VI, VII and VIII.

The invention further provides an immunogenic composition comprising a polypeptide, or a fragment thereof, which is encoded by a polynucleotide sequence which is specific to one or more clinical isolates of a Streptococcal species. Preferably, the polynucleotide is specific to a Streptococcal species clinical isolate selected from the Streptococcal species GBS, GAS and pneumococcus. More preferably, the polynucleotide is specific to one or more GBS clinical isolates selected from the clinical isolates identified in Table 5. Still more preferably, the polynucleotide is specific to one or more GBS clinical isolates having one or more genes selected from the genes listed in Table 7.

In another embodiment, the polynucleotide is a GBS polynucleotide which is homologous to at least one gene from both GAS and pneumococcus and which varies among clinical isolates. In another embodiment, the polynucleotide is a GBS polynucleotide which is homologous to at least one gene from both GAS and pneumococcus and which is homologous with at least one gene from at least one of the clinical isolates identified in Table 5. In another embodiment, the polynucleotide is a GBS polynucleotide which is homologous to at least one gene from both GAS and pneumococcus and which is homologous with at least one gene from each of the clinical isolates identified in Table 5. Preferably, the polynucleotide is selected from one of the genes listed in Table 7.

In one embodiment, the polynucleotide is a GBS polynucleotide which is homologous to at least one gene from GAS and is not homologous to any gene from pneumococcus and which varies among clinical isolates. In another embodiment, the polynucleotide is a GBS polynucleotide which is homologous to at least one gene from GAS and is not homologous to any gene from pneumococcus and which is homologous to at least one gene from at least one of the clinical isolates identified in Table 5. In another embodiment, the polynucleotide is a GBS polynucleotide which is homologous to at least one gene from GAS and is not homologous to any gene from pneumococcus and which is homologous to at least one gene from each of the clinical isolates identified in Table 5. Preferably, the polynucleotide is selected from one of the genes listed in Table 7.

In one embodiment, the polynucleotide is a GBS polynucleotide which is homologous to at least one gene from pneumococcus and is not homologous to any gene from GAS and which varies among clinical isolates. In another embodiment, the polynucleotide is a GBS polynucleotide which is homologous to at least one gene from pneumococcus and is not homologous to any gene from GAS and which is homologous to at least one gene from at least one of the clinical isolates identified in Table 5. In another embodiment, the polynucleotide is a GBS polynucleotide which is homologous to at least one gene from pneumococcus and is not homologous to any gene from GAS and which is homologous to at least one gene from each of the clinical isolates identified in Table 5. Preferably, the polynucleotide is selected from one of the genes listed in Table 7.

In one embodiment, the polynucleotide is a GBS polynucleotide which is not homologous to any gene from GAS or pneumococcus and which varies among clinical isolates. In another embodiment, the polynucleotide is a GBS polynucleotide which is not homologous to any gene from GAS or pneumococcus and which is homologous to at least one gene from at least one of the clinical isolates identified in Table 5. In another embodiment, the polynucleotide is a GBS polynucleotide which is not homologous to any gene from GAS or pneumococcus and which is homologous to at least one gene from each of the clinical isolates identified in Table 5. Preferably, the polynucleotide is selected from one of the genes listed in Table 7.

The invention further provides an immunogenic composition comprising a polypeptide, or a fragment thereof, which is encoded by a polynucleotide sequence which is conserved across one or more clinical isolates of a Streptococcal species. Preferably, the polynucleotide is conserved across one or more Streptococcal clinical isolates selected from the Streptococcal species GBS, GAS and pneumococcus. More preferable, the polynucleotide is conserved across one or more GBS clinical isolates identified in Table 5. Still more preferably, the polynucleotide is conserved across one or more clinical isolates having one or more genes selected from the genes listed in Table 7.

The invention further provides for an immunogenic composition comprising a polypeptide, or a fragment thereof, encoded by a polynucleotide selected from one or more of the Subsets of the invention. Accordingly, the invention provides for an immunogenic composition comprising a polypeptide encoded by a polynucleotide selected from one or more of the following Subsets: GBS Subset 1, GBS Subset 2, GBS Subset 3, GBS Subset 4, GAS Subset 1, GAS Subset 2, GAS Subset 3, GAS Subset 4, Spn Subset 1, Spn Subset 2, Spn Subset 3, Spn Subset 4, GBS Subset l(a), GBS Subset 2(a), GBS Subset 3(a), GBS Subset 4(a), GAS Subset 1(a), GAS Subset 2(a), GAS Subset 3(a), GAS Subset 4(a), Spn Subset 1(a), Spn Subset 2(a), Spn Subset 3(a), Spn Subset 4(a), GBS Subset 1(b), GBS Subset 2(b), GBS Subset 3(b), GBS Subset 4(b), GBS Subset 5, GBS Subset 6, GBS Subset 6(a), GBS Subset 7, GBS Subset 8, GBS Subset 9, GBS Subset 10, GBS Subset 11, GBS Subset 12, GBS Subset 12(a), GBS Subset 12(b), GBS Subset 12(c), GBS Subset 12(d), GBS Subset 12(e), GBS Subset 12(f), GBS Subset 12(g), GBS Subset 12(h), GBS Subset 12(i), GBS Subset 12(j), GBS Subset 12(k), GBS Subset 12(l), GBS Subset 12(m), GBS Subset 12(n), GBS Subset 12(o), GBS Subset 13(a), GBS Subset 13(b), GBS Subset 13(c), GBS Subset 13(d), GBS Subset 13(e), GBS Subset 13(f), GBS Subset 13(g), GBS Subset 13(h), GBS Subset 13(i), GBS Subset 13(j), GBS Subset 13(k), GBS Subset 13(l), GBS Subset 13(m), GBS Subset 13(n), GBS Subset 13(o), GBS Subset 13(p), GBS Subset 13(q), GBS Subset 14, GBS Subset 14(a), GBS Subset 14(b), GBS Subset 14(c), GBS Subset 14(d), GBS Subset 14(e), GBS Subset 14(f), GBS Subset 14(g), and GBS Subset 14(h).

The invention provides for an immunogenic composition comprising a polypeptide, or a fragment thereof, encoded by a polynucleotide selected from one or more of the following Subsets: GBS Subset 1, GBS Subset 2, GBS Subset 3, and GBS Subset 4.

The invention provides for an immunogenic composition comprising a polypeptide, or a fragment thereof, encoded by a polynucleotide selected from one or more of the following Subsets: GAS Subset 1, GAS Subset 2, GAS Subset 3, and GAS Subset 4.

The invention provides for an immunogenic composition comprising a polypeptide, or a fragment thereof, encoded by a polynucleotide selected from one or more of the following Subsets: Spn Subset 1, Spn Subset 2, Spn Subset 3, and Spn Subset 4.

The invention provides for an immunogenic composition comprising a polypeptide, or a fragment thereof, encoded by a polynucleotide selected from one or more of the following Subsets: GBS Subset 1(a), GBS Subset 2(a), GBS Subset 3(a), and GBS Subset 4(a).

The invention provides for an immunogenic composition comprising a polypeptide, or a fragment thereof, encoded by a polynucleotide selected from one or more of the following Subsets: GAS Subset 1(a), GAS Subset 2(a), GAS Subset 3(a), and GAS Subset 4(a).

The invention provides for an immunogenic composition comprising a polypeptide, or a fragment thereof, encoded by a polynucleotide selected from one or more of the following Subsets: Spn Subset 1(a), Spn Subset 2(a), Spn Subset 3(a), and Spn Subset 4(a).

The invention provides for an immunogenic composition comprising a polypeptide, or a fragment thereof, encoded by a polynucleotide selected from one or more of the following Subsets: GBS Subset 1(b), GBS Subset 2(b), GBS Subset 3(b), and GBS Subset 4(b).

The invention provides for an immunogenic composition comprising a polypeptide, or a fragment thereof, encoded by a polynucleotide selected from GBS Subset 5.

The invention provides for an immunogenic composition comprising a polypeptide, or a fragment thereof, encoded by a polynucleotide selected from one or more of the following Subsets: GBS Subset 6 and GBS Subset 6(a).

The invention provides for an immunogenic composition comprising a polypeptide, or a fragment thereof, encoded by a polynucleotide selected from one or more of the following Subsets: GBS Subset 7.

The invention provides for an immunogenic composition comprising a polypeptide, or a fragment thereof, encoded by a polynucleotide selected from one or more of the following Subsets: GBS Subset 8.

The invention provides for an immunogenic composition comprising a polypeptide, or a fragment thereof, encoded by a polynucleotide selected from one or more of the following Subsets: GBS Subset 9.

The invention provides for an immunogenic composition comprising a polypeptide, or a fragment thereof, encoded by a polynucleotide selected from one or more of the following Subsets: GBS Subset 10.

The invention provides for an immunogenic composition comprising a polypeptide, or a fragment thereof, encoded by a polynucleotide selected from one or more of the following Subsets: GBS Subset 11.

The invention provides for an immunogenic composition comprising a polypeptide, or a fragment thereof, encoded by a polynucleotide selected from one or more of the following Subsets: GBS Subset 12, GBS Subset 12(a), GBS Subset 12(b), GBS Subset 12(c), GBS Subset 12(d), GBS Subset 12(e), GBS Subset 12(f), GBS Subset 12(g), GBS Subset 12(h), GBS Subset 12(i), GBS Subset 12(j), GBS Subset 12(k), GBS Subset 12(l), GBS Subset 12(m), GBS Subset 12(n), and GBS Subset 12(o).

The invention provides for an immunogenic composition comprising a polypeptide, or a fragment thereof, encoded by a polynucleotide selected from one or more of the following Subsets: GBS Subset 13(a), GBS Subset 13(b), GBS Subset 13(c), GBS Subset 13(d), GBS Subset 13(e), GBS Subset 13(f), GBS Subset 13(g), GBS Subset 13(h), GBS Subset 13(i), GBS Subset 13(j), GBS Subset 13(k), GBS Subset 13(l), GBS Subset 13(m), GBS Subset 13(n), GBS Subset 13(o), GBS Subset 13(p), GBS Subset 13(q).

The invention provides for an immunogenic composition comprising a polypeptide or a fragment thereof encoded by a polynucleotide selected from one or more of the following Subsets: GBS Subset 14, GBS Subset 14(a), GBS Subset 14(b), GBS Subset 14(c), GBS Subset 14(d), GBS Subset 14(e), GBS Subset 14(f), GBS Subset 14(g), and GBS Subset 14(h).

Each of the above-identified groups and subsets may be used to create immunogenic compositions comprising two or more Streptococcus polypeptides. The invention then provides for an immunogenic composition comprising a combination of Streptococcus polypeptides, said combination consisting of two, three, four, five, six, seven, eight, nine, or ten polypeptides selected from one of the groups identified above. Preferably, the combination consists of two, three, four or five polypeptides. Preferably, the polypeptides are all selected from the same group. Preferably, the polypeptides are selected from the same Subset described herein. The Streptococcus polypeptides are selected from GBS, GAS and pneumococcus. Preferably, all of the polypeptides in the combination are selected from the same species.

For example, the composition may comprise an combination of GBS polypeptides, said combination consisting of two, three, four, five, six, seven, eight, nine, or ten polypeptides, wherein each polypeptide is encoded by a GBS polynucleotide sequence which is homologous to a polynucleotide sequence of both GAS and pneumococcus. Preferably, the combination consists of two, three, four or five polypeptides. Preferably, the GBS polynucleotide sequences are selected from GBS Subset 1.

As another example, the composition may comprise a combination of GBS polypeptides, said combination consisting of two, three, four or five polypeptides, wherein each polypeptide is encoded by a GBS polynucleotide sequence which is homologous to a polynucleotide sequence of GAS. Preferably, the GBS polynucleotide sequences are selected from GBS Subset 2.

The composition may comprise a combination of GBS polypeptides, said combination consisting of two, three, four or five polypeptides, wherein each polypeptide is encoded by a GBS polynucleotide sequence which is homologous to a polynucleotide sequence of Streptococcus pneumoniae. Preferably, the GBS polynucleotide sequences selected from GBS Subset 3.

The composition may comprise a combination of GBS polypeptides, said combination consisting of two, three, four or five polypeptides, wherein each polypeptide is encoded by a GBS serotype polynucleotide sequence which is homologous to at least one other GBS serotype. Preferably, the GBS polypeptides are encoded by GBS serotype polynucleotide sequences which are homologous to at least one other GBS serotype.

The invention further provides for an immunogenic composition comprising a polypeptide or a fragment thereof comprising a fusion protein encoded by one or more of the polynucleotides included in the Subsets of the invention.

The invention further provides a method for designing an immunogenic composition, such as a vaccine, by selecting one or more polypeptides encoded by a polynucleotide selected from one or more of the Subsets of the invention. Preferably, the immunogenic compositions of the invention comprise at least two, three, four or five polypeptides encoded by polynucleotides within the same Subset.

The invention provides a method for raising an immune response in a patient by administering any one of the immunogenic compositions set forth above. The choice of immunogenic composition means that the immune response may be reactive against all three of GAS, GBS and streptococcus, may be reactive against only two of the three, or may be reactive only against GBS.

Each of the immunogenic compositions described above may be prepared and administered instead as a polynucleotide where the polypeptide is expressed in vivo.

The immune response is preferably an antibody response. It may be a protective immune response. The patient is preferably a human.

The immunogenic compositions of the invention may further comprise an adjuvant, as discussed in further detail below.

### Essential Genes and Knockouts

The invention provides a Streptococcus bacterium wherein one or more genes within any of the Subsets of this invention have been knocked out. The choice of Subset means that the knocked out gene may be, for instance, a gene found in GBS but not in GAS or pneumococcus (e.g. which is involved in the pathogenesis of GBS, but not in the pathogenesis of GAS or pneumococcus, such as binding GBS cellular targets).

Techniques for producing knockout bacteria are well known, and knockout Streptococci of various species have been reported [e.g. Margolis et al. (2001) Antimicrob. Agents Chemother. 45:2432-2435; Zhang et al. (2000) Cell 102:827-837; Nizet et al. (2000) Infect. Immun. 68:4245-4254; Nizet et al. (1997) Adv. Exp. Med. Biol. 418:627-630; etc.].

The knockout mutation may be situated in the coding region of the gene or may lie within its transcriptional control regions (e.g. within its promoter).

The knockout mutation will reduce the level of mRNA encoding the corresponding polypeptide to <1% of that produced by the wild-type bacterium, preferably <0.5%, more preferably <0.1%, and most preferably to 0%.

The knockout mutants of the invention may be used as immunogenic compositions (e.g. as vaccines) to prevent streptococcal infection. Such a vaccine may include the mutant as a live attenuated bacterium.

The knockout mutants of the invention may be used to determine whether genes are essential for bacterial survival, either under normal or stress conditions.

### Antisense

The invention provides a single-stranded nucleic acid comprising a fragment of x₁ or more nucleotides from a nucleotide sequence selected from one of the Subsets of the invention. The choice of group means that the nucleic acid may be complementary to a gene sequence found in GBS, GAS and pneumococcus, or a gene sequence specific to GBS.

The single-stranded nucleic acid is at least x₁ nucleotides long. The value of x₁ is at least 7 (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 etc.). The single-stranded nucleic acid may be at most x₂ nucleotides long, wherein x₂ is 100 or less (e.g. 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60).

The nucleic acid is preferably of the formula 5′(N)ₐ—(X)—(N)_{b}-3′, wherein 0≧a≧15, 0≧b≧15, N is any nucleotide, and X is the fragment as defined above. The values of a and b may independently be 0, 1, 2, 3, 4, 5, 6, 7, 8, 9, 10, 11, 12, 13, 14 or 15. Each individual nucleotide N in the —(N)ₐ— and —(N)_{b}— portions of the nucleic acid may be the same or different. The length of the nucleic acid (i.e. a+b+x₁) is preferably x₂ or less.

Antisense inhibition of streptococcal gene expression is known e.g. Sato et al. (1998) FEMS Microbiol Lett 159:241-245. Antibacterial antisense techniques are also disclosed in international patent applications WO99/02673 and WO99/13893.

The single-stranded nucleic acid may reduce the level of polypeptide expression from the complementary gene to <1% of that produced by the wild-type bacterium, preferably <0.5%, more preferably <0.1%, and most preferably to 0%.

Antisense experiments may be used to determine whether genes are essential for bacterial survival, either under normal or stress conditions.

### Screening Methods

The invention provides a method for screening compounds, wherein the method involves contacting the compounds with a polypeptide expressed by one or more of the polynucleotides selected from one of the Subsets of the invention. The method may be for screening for agonists of the polypeptides, antagonists, antibiotics etc. The choice of group means, for instance, that the method may be used for identifying an antibiotic with broad anti-streptococcal activity could be identified, or for identifying an antibiotic specific to GBS.

Potential compounds for screening include small organic molecules, peptides, peptoids, polypeptides, lipids, metals, nucleotides, nucleosides, aptamers, polyamines, antibodies, and derivatives thereof. Small organic molecules have a molecular weight between 50 and about 2,500 daltons, and most preferably in the range 200-800 daltons. Complex mixtures of substances, such as extracts containing natural products, compound libraries or the products of mixed combinatorial syntheses also contain potential antagonists.

Typically, a polypeptide is incubated with a test compound, and the mixture is then tested to see if the polypeptide and test compound interact, or to see if the polypeptide's activity is inhibited.

For preferred high-throughput screening methods, all the biochemical steps for this assay are performed in a single solution in, for instance, a test tube or microtitre plate, and the test compounds are analysed initially at a single compound concentration. For the purposes of high throughput screening, the experimental conditions are adjusted to achieve a proportion of test compounds identified as “positive” compounds from amongst the total compounds screened.

The invention also provides a compound identified using these methods. These can be used to treat or prevent streptococcal infection. The compound preferably has an affinity for the adhesion-specific protein of at least 10⁻⁷ M e.g. 10⁻⁸ M, 10⁻⁹ M, 10⁻¹⁰ M or tighter.

### Distinguishing Streptococcal Species

The invention provides a method for determining whether a Streptococcus bacterium of interest is or is not in the species agalactiae, pyogenes or pneumoniae, comprising the step(s) of: (a) contacting the bacterium with a nucleic acid probe comprising the sequence of a gene selected from one of the Subsets of the invention; and/or (b) contacting the bacterium with an antibody which binds to a polypeptide encoded by one or more of the polynucleotides of one or more of the Subsets of the invention. The choice of group means, for instance, that the method may be used for distinguishing GBS from GAS and from pneumococcus, or for confirming that a bacterium is not a GAS or pneumococcus.

The method will typically include the further step of detecting the presence or absence of an interaction between the bacterium of interest and the nucleic acid or protein.

The bacterium of interest may be in a cell culture, for example, or may be within a biological sample believed or known to contain a streptococcus. It may be intact or may be, for instance, lysed.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

### GBS 2603 Type V Genomic Sequence

Applicants have sequenced the complete genome sequence of GBS clinical type V isolate 2603 V/R and performed comparative analyses comparing this sequence with other GBS strains, with other species of pathogenic Streptococci and with other known bacterial species. The entire genomic sequence is available by Aug. 26, 2002 at http://www.tigr.org. This genomic sequence is incorporated herein by reference in its entirety. The genomic sequence of GBS type V isolate 2603 V/R is also set forth in International Patent Application WO 02/34771.

In one embodiment, the invention relates to the polynucleotides, and fragments and derivatives thereof, set forth in the GBS clinical type V isolate 2603 published genome which are not disclosed within WO 02/34771. The invention further relates to polypeptides expressed by the polynucleotides of the invention.

Applicants have predicted that the GBS 2603 isolate contains approximately 2,176 predicted genes. Each predicted gene is set forth in Table 1, listed by a SAGxxxx ORF number. Table 1 also includes the predicted amino acid size of the predicted expressed protein and the predicted function, if known. The sequence of each SAG reference can be obtained at the TIGR website.

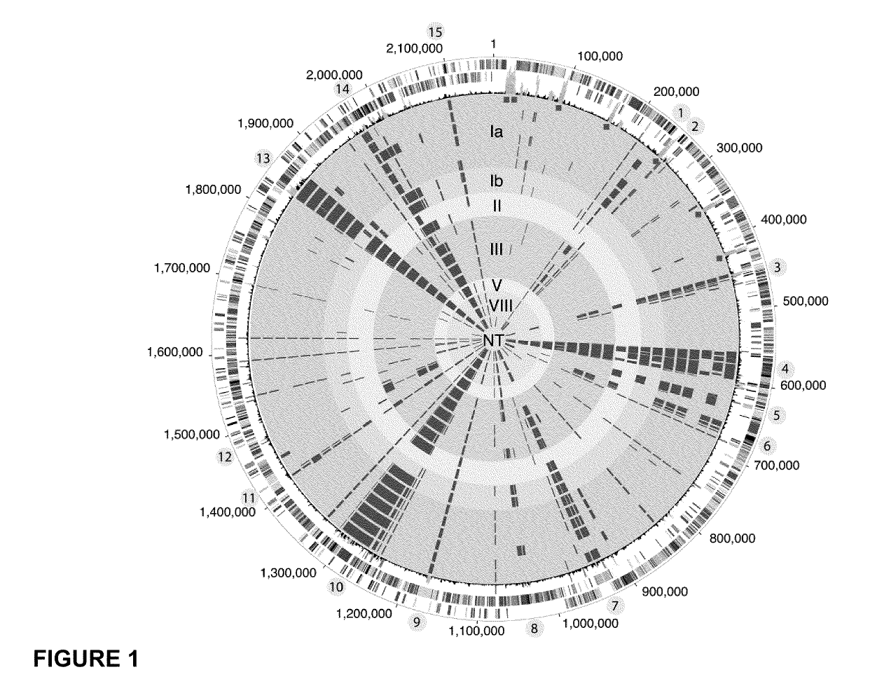

FIG. 1 is a circular representation of the GBS genome and comparative hybridisations using microarrays. A color version of FIG. 1 can be found in Tettelin et al., PNAS (2002) 99(19): 12391-12396 and online at www.pnas.org. The outer circle represents predicted coding regions on the plus strand color coded by role categories: violet indicating amino acid biosynthesis; light blue indicating biosynthesis of cofactors, prosthetic groups, and carriers; light green indicating cell envelope; red indicating cellular processes; brown indicating central intermediary metabolism; yellow indicating DNA metabolism; light gray indicating energy metabolism; magenta indicating fatty acid and phospholipid metabolism; pink indicating protein synthesis and fate; orange indicating purines, pyrimidines, nucleosides, and nucleotides; olive indicating regulatory functions and signal transduction; dark green indicating transcription; teal indicating transport and binding proteins; gray indicating unknown function; salmon indicating other categories; blue indicating hypothetical proteins.

The second circle represents predicted coding regions on the minus strand. In the third circle, black represents atypical nucleotide composition curve; green represents most atypical regions; magenta represents insertion elements; red diamonds indicate rRNAs.

Circles 4-22 represent comparative hybridisations of strain 2603 V/R with 19 GBS strains. Cy3/Cy5 (2603 V/R signal/test strain) ratio cutoffs were defined arbitrarily as Cy3/Cy5—1.0-3.0, the gene was present in the test strain, no color was added; Cy3/Cy5=3.0-10.0, ambiguous result (blue); Cy3/Cy5>10, gene absent in test strain (red).

Circles 4-9 represent type 1a strains 090, 515, A909, Davis, and DK8. Circles 10-11 represent type 1b strains S7 7357b and H36B. Circles 12-13 represent type II strains 18RS21 and DK21. Circles 14-18 represent type III COH1, COH31, D136C, M732 and M781. Circle 19 represents type V strain CJB111. Circles 20-21 represent type VIII strains SMU014 and JM9130013. Circle 22 represents nontypable (NT) strain CJB110. Throughout FIG. 1, varying regions of five or more consecutive genes are indicated by yellow bullets.

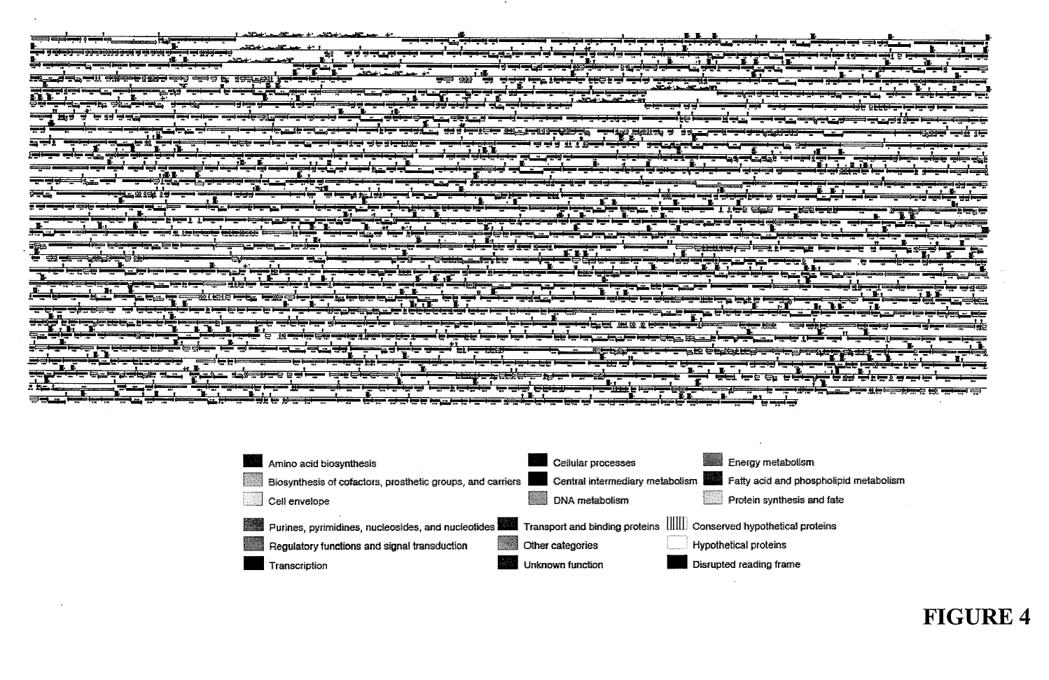

FIG. 4 depicts a linear representation of the GBS genome. The location of predicted coding regions color-coded by biological role (see FIG. 1) is displayed. Arrowed boxes represent the direction of transcription for each ORF. The number of membrane-spanning domains predicted by TopPred is displayed as lipid bi-layers on top of ORFs, only for those whose products have five or more predicted membrane spanning regions. Genes coding for rRNAs (16S,23S,5S) and tRNAs (clover leaf structure with number of genes) are indicated. Predicted Rho-independent transcriptional terminators are represented by hairpins.

ORF's were predicted by GLIMMER (See, Delcher, et al., (1999) Nucleic Acids Res. 27, 4636-4641 and Salzberg, et al., (1998) Nucleic Acids Res. 26, 544-548) trained with ORFs larger than 600 base pairs from the genomic sequence and GBS genes available in GenBank. All predicted proteins larger than 30 amino acids were searched against a nonredundant protein database. (See Fleischmann, et al., (1995) Science 269, 496-512). Frame-shifts and point mutations were detected and corrected where appropriate; those remaining were annotated as “authentic frame-shift” or “authentic point mutation”. Protein membrane-spanning domains were identified by TOPPRED (See Claros, et al., (1994) Comput. Appl. Biosci. 10, 685-686). Candidate lipoprotein signal peptides (See Hayashi et al., (1990) J. Bioenerg. Biomembr. 22, 451-471) were flagged by N-terminal exact matches to the pattern {DERK} (6)-[LIVMFWSTAG] (2)-[LIVMFYSTAGCQ]-[AGS]-C. Putative signal peptides were identified by using SIGNALP (Nielsen, et al., (1997) Protein Eng. 10, 1-6). Two sets of hidden Markov models were used to determine ORF membership in families and superfamilies: PFAM Ver. 5.5 (Bateman, et al., (2000) Nucleic Acids Res. 28, 263-266) and TIGRFAMS 1.0 (Haft et al., (2001) Nucleic Acids Res. 29, 41-43). Domain-based paralogous families were built by performing all-versus-all searches on the protein sequences by using a modified version of a previously described method. (Niermann, et al., (2001) Proc. Natl. Acad. Sci. USA 98, 4136-4141) Potential lineage-specific gene duplications were estimated by identification of OFRs more similar to ORFs within the GBS genome than to ORFs from other complete genomes. All ORFs were searched with FASTA3 (Pearson (2000) Methods Mol. Biol. 132, 185-219) against all ORF's from the complete genomes and matches with a FASTA P value of 10⁻¹⁵ were considered significant.

The genome consists of a circular chromosome of 2,160,266 base pairs with a G+C content of 35.7%. Base pair one of the chromosome was assigned within the putative origin of replication. The genome contains 80 tRNAs, 7rRNAs, and 3 sRNAs. Approximately 78% of the 2,176 predicted genes are transcribed in the same direction as that of DNA replication, a feature also observed in S. pn. and other low-GC Gram positive organisms.

Biological roles were assigned to 1,409 (65%) of the genome according to a classification scheme adapted from Riley (1993) Microbiol. Rev. 57, 862-952. Another 527 predicted proteins (24%) matched proteins of unknown function, and the remaining 240 (11%) had no database match. The expression of 50 of these hypothetical proteins was confirmed by Western Blot analysis, and the proteins were annotated as “proteins of unknown function.” A total of 339 paralogous protein families were identified in strain 2603, containing 941 predicted proteins (43% of the total).

The Western Blot analysis was conducted as follows. GBS strain 2603 V/R cells were grown in Todd-Hewitt broth (Difco) to OD600nm=0.5. The culture was centrifuged for 20 minutes at 5,000 rpm. The supernatant was discarded, and bacteria were washed once with PBS, resuspended in 2 ml of 50 mM Tris-HCl pH 6.8, containing 400 units of Mutanolysin (Sigma), and incubated 2 hours at 37° C. After three cycles of freeze and thaw, cellular debris was removed by centrifugation at 14,000 rpm for 10 minutes, and the protein concentration of the supernatant was measured by the Bio-Rad Protein assay, with BSA as a standard. Purified recombinant proteins (50 ng) and total cell extracts (25 μg) derived from GBS serotype V 2603 V/R strain were separated by SDS/PADE and electroblotted onto nitrocellulose membranes for 1 hour at 100 V. The membranes were saturated by overnight incubation at 4° C. in 5% skimmed milk and 0.1% Tween 20 in PBS and incubated for 1 hour at room temperature with sera from immunized mice diluted 1:500-1:1,000 in saturation buffer. To reduce background due to antibodies raised against contaminating E. coli proteins, sera were preincubated with E. coli protein extracts absorbed on nitrocellulose strips. The membranes were washed twice in 3% skimmed milk and 0.1% Tween 20 in PBS and incubated for 1 hour with a 1:1,000 dilution of horseradish peroxidase-conjugated antimouse Ig (DAKO). After washing with 0.1% Tween 20 in PBS, the membranes were developed with the Opti-4CN Substrate Kit (Bio-Rad).

Table 2 comprises a list of predicted and experimentally characterized surface and secreted proteins from GBS. Candidate signal peptides and lipoprotein motifs were predicted with PSORT [Nakai, K & Horton, P. (1999) Trends Biochem Sci 24, 34-6 ] and other methods (see methods), sortase motifs (LPxTG) were detected using the FINDPATTERNS program of the GCG Package [Devereux, J., Haeberli, P. & Smithies, O. (1984) Nucleic Acids Res 12, 387-95] and hidden Markov models. Column “Other” indicates proteins carrying other motifs (e.g. integrin-binding motif RGD) or are similar to characterized surface-exposed proteins. Western blot results were considered positive when the antibodies revealed a predominant band of the expected molecular weight on the total protein extracts of S. agalactiae strain 2603 V/R, ORFs without + or − in this column were not tested in western blot. FACS analyses were performed for western blot positive proteins only. Western blot and FACS data are displayed only for proteins carrying at least one of the other motifs shown in the table. Column “GBS specific” indicates genes unique to S. agalactiae (when compared to other completely sequenced genomes) that are present in all the S. agalactiae strains tested in comparative genome hybridization analyses. Finally, only proteins carrying less than 3 predicted transmembrane domains are shown in the table, other proteins are likely to be embedded in the cytoplasmic membrane and are probably not exposed on the organism's surface.

FACS data was collected as follows: GBS 2603 V/R strain cells were grown in Todd-Hewitt broth (Difco) to OD600nm=0.5. The culture was centrifuged for 20 minutes at 5,000 rpm, and bacteria were washed once with PBS, resuspended in PBS containing 0.05% paraformaldehyde, and incubated for 1 hour at 37° C. and then overnight at 4° C. Fifty microliters of fixed bacteria (OD600nm 0.1) was washed once with PBS, resuspended in 20 μl of newborn calf serum (Sigma), and incubated for 1 hour at 4° C. in 100 μl of preimmune or immune sera and diluted 1:200 in dilution buffer (PBS, 20% newborn calf serum, 0.1% BSA). After centrifugation and washing with 200 μl of washing buffer (0.1% BSA in PBS), samples were incubated for 1 hour at 4° C. with 50 μl of R-phycoeryrin-conjugated F(ab)₂ goat anti-mouse IgG (Jackson ImmunoResearch) diluted 1:100 in dilution buffer. Cells were washed with 200 μl of washing buffer and resuspended in 200 μl of PBS. Samples were analysed by using a FACS calibur apparatus (Becton Dickinson), and data were analyzed by using CELL QUEST (Becton Dickinson). A shift in mean fluorescence intensity of >75 channels compared with preimmune sera from the same mice was considered positive. This cutoff was determined from the mean plus two standard deviations of shifts obtained with control sera raised against mock purified recombinant proteins from cultures of E. coli carrying the empty expression vector and included in every experiment. Artifacts due to bacterial lysis were excluded by using antisera raised against six different known cytoplasmic proteins, all of which gave negative results.

### Regions of Atypical Nucleotide Composition.

These regions were identified by the x² analysis: the distribution of all 64 trinucleotides (3 mers) was computed for the complete genome in all six reading frames, followed by the 3-mer distribution in 2,000-bp windows. Windows overlapped by 1,000 bp. For each window, the x² statistic on the difference between its 3-mer content, and that of the whole genome was computed.

### In Silico Genome Comparisons

The protein sets of S. agalactiae, Streptococcus pneumoniae and S. pyogenes were compared by using FASTA3. A general description of the FASTA3 sequence comparison program is discussed in Pearson, W.R., “Flexible Sequence Similarity Searching with the FASTA3 Program Package”, (2000) Methods Mol. Biol., 132: 185-219. Shared genes were defined using a FASTA3 P value cutoff of 10⁻¹⁵. These shared genes and genes that S. agalactiae did not share with the other streptococci using this cutoff were subsequently searched against all completely sequenced genomes, and genes were defined as unique to streptococci or S. agalactiae when they did not share similarity with any other gene sets with a FASTA3 P value of 10⁻⁵ or lower. The use of two cutoffs provides for a more stringent analysis of shared or unique genes.

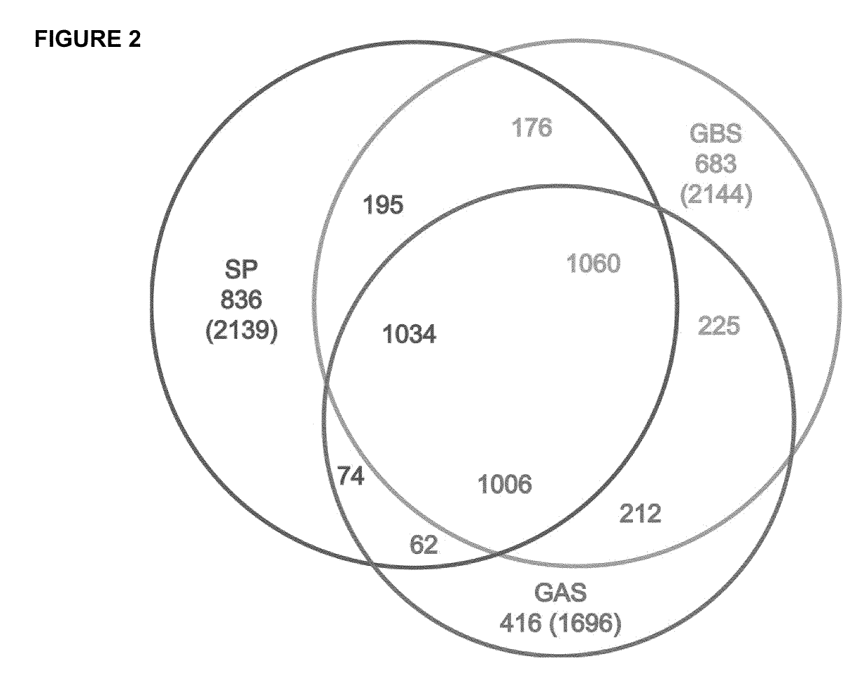

FIG. 2 is a schematic representation of in silico comparisons between streptococci. The protein sets of GBS, S. pn., and GAS were compared by using FASTA3. Numbers under the species name indicate genes that are not shared with the other species; values in parenthesis are the number of proteins in each species (excluding frame-shifted and degenerated genes). Numbers in the intersections indicate genes shared by two or three species. These are displayed in the color corresponding to the species used as the query. (GBS: green; S.pn.: blue; GAS: red. A color version of FIG. 2 can be found in Tettelin et al., PNAS (2002) 99(19): 12391-12396 and online at www.pnas.org.). Numbers in any given intersection are slightly different due to gene duplications in some species.

Table 3 lists genes which were shared among GBS, GAS and pneumococcus, but which were not found in any of the other completely sequenced genomes. The protein sets of S. agalactiae, S. pneumoniae, and S. pyogenes were compared using FASTA3 [Pearson, W. R. (2000) Methods Mol Biol 132, 185-219]. Shared genes were defined using a FASTA3 p value cutoff of 10⁻¹⁵. These shared genes and genes that S. agalactiae did not share with the other streptococci using this cutoff were subsequently searched against all completely sequenced genomes and genes were defined as unique to streptococci or S. agalactiae when they did not share similarity with any other gene sets with a FASTA3 p value of 10⁻⁵ or lower.

### Synteny

Regions of conservation of gene synteny were computed as windows of 10 kb spanning at least three genes whose order was conserved in the other species. Regions were merged if they were less than 20 kb apart. The number of genes within each broad region was then calculated.

### Comparative Genome Hybridizations

Comparative genome hybridizations (See FIG. 1) using DNA microarrays were performed between the sequenced type V strain 2603 V/R and 19 other GBS strains of multiple serotypes (See Table %). Predicted genes from strain 2603 V/R were amplified by PCR and arrayed on glass microscope slides. See Peterson, et al., (2000) J. Bacteriol. 182, 6192-6202. Genomic DNA was labelled according to protocols provided by J. DeRisi (www.microarrays.org/Pdfs/Genomic-DNALabel_B.pdf), except that the DNA was not digested or sheared before labelling. Arrays were scanned with a GENEPIX 4000B scanner (Axon Instruments, Foster City, Calif.), and individual hybridisation signals were quantitated with TIGR SPOTFINDER. See Hedge, et al., (2000), Biotechniques 29, 548-550, 552-554, 556. Cy3/Cy5 (2603 V/R signal/test strain) ratio cutoffs were defined arbitrarily as Cy3/Cy5=1.0-3.0, gene present in test strain; 3.0-10.0, ambiguous result; >10.0, gene absent. For ambiguous results, the gene may be divergent in the test strain relative to 2603 V/R, or the gene may be absent in the test strain but still produces paralogous gene family or a repetitive element. Although cutoffs are arbitrary, they fit nicely the results for the variation of the capsule locus in the strains tested (see region 9 on FIG. 1) where most genes are slightly divergent and only a few are completely different.

The CGH detected 1,698 genes in all of the strains, whereas 401 genes from strain 2603 V/R (18% of the gene complement) were not detected in at least one other strain, suggesting that they are absent or significantly divergent in those strains. Two hundred sixty (38%) of the 683 genes specific to S. agalactiae when compared with the other two streptococci (FIG. 2), including virulence determinants and surface proteins, vary among S. agalactiae strains, whereas only 47 (4%) of the genes common to all three streptococcal species, including 5 of the 6 sortases identified in the genome, vary among strains. Thus, the in silico analysis of genes shared by the streptococci that are not expected to vary among this genus is consistent with the CGH analysis. Forty-four (25%) of the genes shared by S. agalactiae and S. pneumoniae and 44 (20%) of those shared by S. agalactiae and S. pyogenes vary in the CGH analysis. The first set contains many glycosyl transferases and proteins carrying a cell-wall anchor, whereas the second set displays many phage-related genes. One hundred thirty-six of the 315 genes unique to S. agalactiae when compared with all sequenced genomes vary among strains. These include R5, three capsular genes, two cell wall-anchored proteins, and three transcriptional regulators. Three hundred sixty-four (91%) of the 401 varying genes correspond to 15 regions containing more than 5 contiguous genes. Ten of these regions display an atypical nucleotide composition in strain 2603 V/R (FIG. 1), consistent with the possibility that they were horizontally transferred into this strain. Two of the largest regions (region 4, a prophage and region 7, similar to Tn916 from Enterococcus faecalis) are flanked by insertion sequence elements. The 15 regions contain many proteins predicted to be anchored on the cell wall or surface exposed, including Rib (region 3), sortases, glycosyl transferases, the capsule locus (region 9, divergent in all strains but the other type V strain CJBL111), and phage-related genes. Region 14 is unique to S. agalactiae and spans 33 genes (SAG1989-SAG2021), including 25 proteins of unknown function, some of which carry a cell-wall anchor. It is flanked by an ISL3 transposase and displays an atypical nucleotide composition. Region 1, unique to S. agalactiae, is a possible plasmid or remnant of a phage (SAG0218-SAG0238), contains mostly hypothetical proteins, and is flanked by a site-specific recombinase. Region 8 is specific to S. agalactiae, comprises 20 proteins of unknown function (SAG1018-SAG1037), most of which are predicted to be membrane associated or secreted, and displays an atypical nucleotide composition.

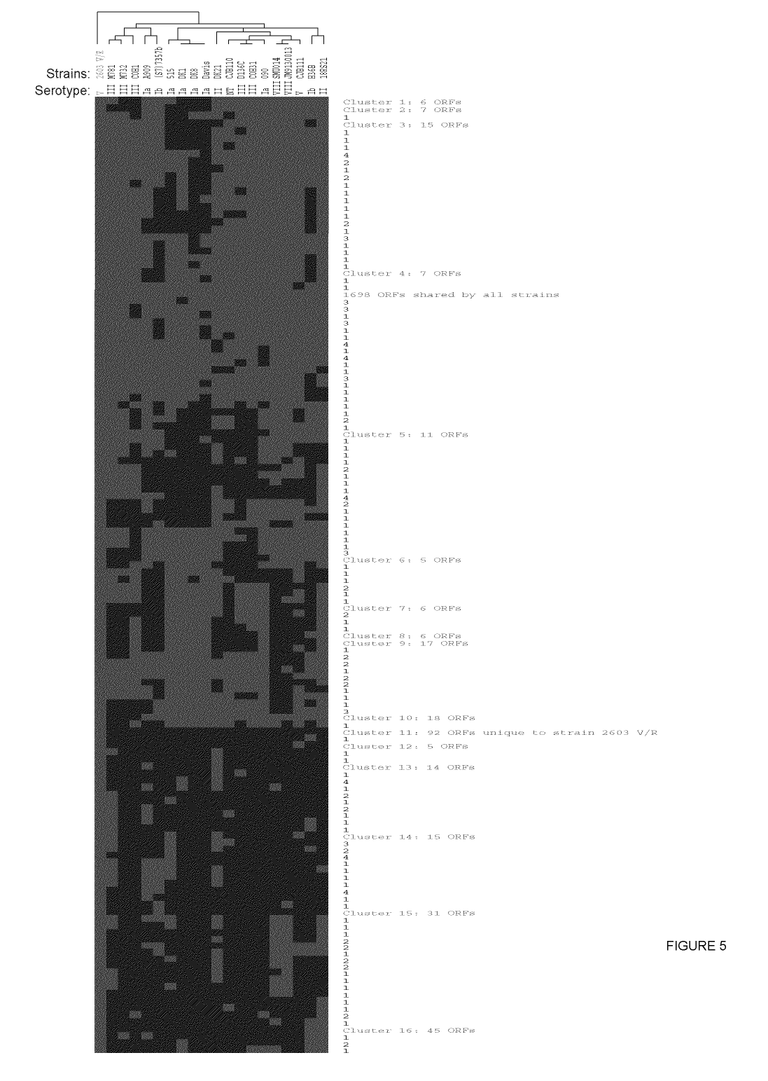

The CGH results were analyzed by profile clustering where genes are grouped based on their distribution patterns (FIG. 5). Sixteen clusters of five or more contiguous and noncontiguous genes comprising a total of 300 genes were identified (Table 6). Several clusters correspond to regions of contiguous genes described above. Some clusters of genes that do not share sequence similarity and are located at different loci in the genome display an identical profile. For instance, a cluster of genes containing a surface antigen (SAG0674-SAG0681) follows the same distribution as another cluster containing only hypothetical proteins (SAG0247-SAG0249). A putative pathogenicity protein (SAG2063) also clusters with a region containing several glycosyl transferases and Sec proteins (SAG1447-SAG1462).

Profile clustering was also used to group strains based on similarity of gene content (FIG. 5). In addition, the sequences of 19 genes from each of 11 S. agalactiae strains were determined after PCR amplification and used for phylogenetic analyses. The strains were the following: type Ia, 090 and A909; type Ib, H36B; type II, 18RS21; type III, COH1, M732 and M781; type V, 2603 V/R and 1169NT1; type VIII, JM9130013; and nontypeable strain CJB110. The set comprised 8 housekeeping genes and 11 genes coding for proteins predicted to be surface-exposed (Table 7).

The profile clustering was conducted as follows. The information and absence of genes based on the comparative genome hybridisation results was used to group genes based on their distribution patterns. The analysis used was essentially identical to that used for phylogenetic profile analysis. See Pellegrinie, et al., (1999) Proc. Natl. Acad. Sci. USA 96, 4285-4288. Each gene was assigned a binary profile based on its presence or absence across the different strains, with presence determined by a Cy3/Cy5 ratio<3.0 and absence≧3.0. The gene profiles were then clustered by using the single-linkage clustering algorithm with column weighting (all with default settings) of CLUSTER (http://rana.lb1.gov). The CLUSTER program also groups the strains (columns) based on similarity of gene profiles. Clusters of genes and strains were viewed by using TREEVIEW (http://rana.lb1.gov).

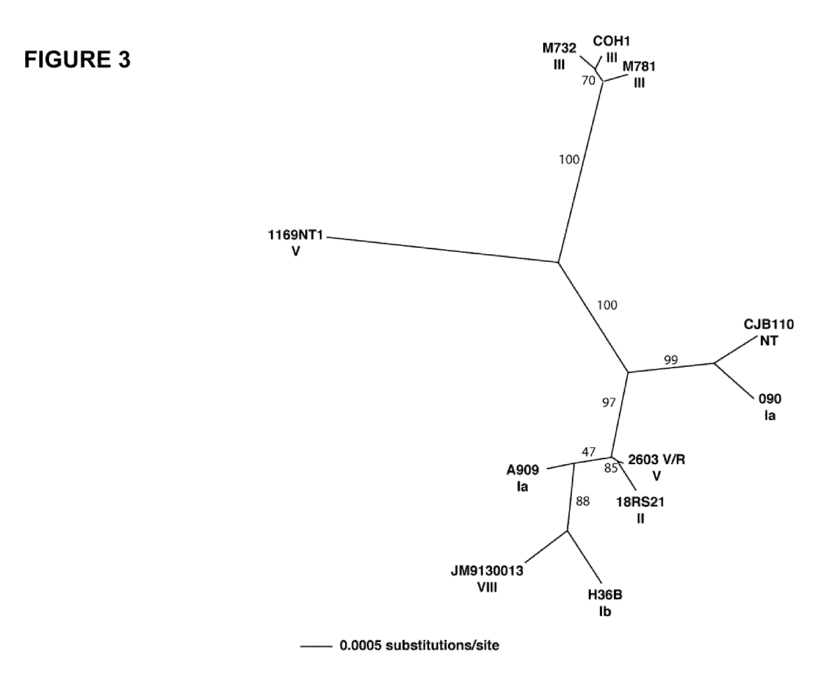

Phylogenetic trees were inferred for the complete set of 19 genes and for the subsets of housekeeping and surface-exposed genes. Because the branching patterns in all three trees were identical, only the tree of the 19 genes is shown in FIG. 3. The degree of polymorphism of the housekeeping and the surface-exposed genes is similar (˜1 variable site among all of the strains per 100 bp).

The sequences of genes from the different strains were aligned by using CLUSTALW (See Thompson (1994), Nucleic Acids Res. 22, 4673-4680.) and trimmed to remove ambiguously aligned regions. Phylogenetic trees of individual genes and of concatenated alignments of multiple genes were inferred by using maximum likelihood methods of PAUP* 4.0 b10 (Sinauer, Sunderland, Mass.). Bootstrap analysis was carried out using PAUP* as well. The possibility of recombination among strains was examined by using analysis of sequence variation using SIMPLOT (S.C. Ray) and analysis of phylogenetic heterogeneity by using MACCLADE (Sinauer).

Analysis of this variation showed no evidence for major recombination events between the strains. There were no long stretches of polymorphic sites that strongly supported other trees (analysis with MACCLADE), and there were no significant crossover events in plots of sequence similarity between strains (analysis with SIMPLOT). Some strain groupings (clades) generated by phylogenetic analysis were similar to clusters from the profile analysis (type III strains M781, M732 and COH1; type Ia strain 090 and nontypable strain CJB110), whereas others were different, possibly because of the aforementioned problems with the profile clustering. In both the phylogenetic analysis and the profile clustering, there is serotypedependent and -independent clustering (FIGS. 3 and 5). The presence of strains of the same serotype in different clades or clusters could be due to lateral gene transfer.

FIG. 5 demonstrates phylogenetic profiling of GBS strains based on comparative genome hybridisations. The information on presence and absence of genes based on the microarray comparative genome hybridization results was used for phylogenetic profile analysis. The presence of a particular gene or gene cluster is indicated in the figure by a red square and the absence of a gene or cluster by a black square. The relationship between strains based on this analysis is depicted by the tree at the top of the figure. The strains and their serotypes are indicated (NT: nontypeable). Clusters with identical profiles are reduced to a single horizontal line and the number of genes in each cluster is indicated on the right. The clusters of 5 or more genes, labeled in red text and numbered, are listed in Table 6. The 1698 genes shared by all 19 strains are labeled in green text.

FIG. 3 depicts a phylogenetic tree of GBS strains based on PCR sequences. The sequences of 19 genes (Table 7) from each of 11 GBS strains were aligned and trimmed to remove ambiguously aligned regions, and phylogenetic trees were inferred. Strain names are indicated in bold, and serotypes are indicated under the strain names. Bootstrap values are indicated on the branches.

### Techniques

A summary of standard techniques and procedures which may be employed in order to perform the invention (e.g. to utilise the disclosed sequences for vaccination or diagnostic purposes) follows. This summary is not a limitation on the invention, but gives examples that may be used, but are not required.

### General

The practice of the present invention will employ, unless otherwise indicated, conventional techniques of molecular biology, microbiology, recombinant DNA, and immunology, which are within the skill of the art. Such techniques are explained fully in the literature e.g. Sambrook Molecular Cloning; A Laboratory Manual, Second Edition (1989) or Third Edition (2000); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986).

Standard abbreviations for nucleotides and amino acids are used in this specification.

### FURTHER DEFINITIONS

A composition containing X is “substantially free of” Y when at least 85% by weight of the total X+Y in the composition is X. Preferably, X comprises at least about 90% by weight of the total of X+Y in the composition, more preferably at least about 95% or even 99% by weight.

The term “comprising” means “including” as well as “consisting” e.g. a composition “comprising” X may consist exclusively of X or may include something additional e.g. X+Y.

The singular forms “a”, “and”, and “the” include plural referents unless the context clearly dictates otherwise. Thus, for example, reference to “a polynucleotide” includes a plurality of such polynucleotides and reference to “an epithelial cell” includes reference to one or more cells and equivalents thereof known to those skilled in the art, etc.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a Streptococcal sequence is heterologous to a mouse host cell. A further examples would be two epitopes from the same or different proteins which have been assembled in a single protein in an arrangement not found in nature.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

A “mutant” sequence is defined as DNA, RNA or amino acid sequence differing from but having sequence identity with the native or disclosed sequence. Depending on the particular sequence, the degree of sequence identity between the native or disclosed sequence and the mutant sequence is preferably greater than 50% (e.g. 60%, 70%, 80%, 90%, 95%, 99% or more, calculated using the Smith-Waterman algorithm as described above). As used herein, an “allelic variant” of a nucleic acid molecule, or region, for which nucleic acid sequence is provided herein is a nucleic acid molecule, or region, that occurs essentially at the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions (e.g. see U.S. Pat. No. 5,753,235).

### Expression Systems

The Streptococcal nucleotide sequences can be expressed in a variety of different expression systems; for example those used with mammalian cells, baculoviruses, plants, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (e.g. structural gene) into mRNA. A promoter will have a transcription initiating region, which is: usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation [Sambrook et al. (1989) “Expression of Cloned Genes in Mammalian Cells.” In Molecular Cloning: A Laboratory Manual, 2nd ed.].

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallotheionein gene, also provide useful promoter sequences. Expression may be either constitutive or regulated (inducible), depending on the promoter can be induced with glucocorticoid in hormone-responsive cells.

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter [Maniatis et al. (1987) Science 236:1237; Alberts et al. (1989) Molecular Biology of the Cell, 2nd ed.]. Enhancer elements derived from viruses may be particularly useful, because they usually have a broader host range. Examples include the SV40 early gene enhancer [Dijkema et al (1985) EMBO J. 4:761] and the enhancer/promoters derived from the long terminal repeat (LTR) of the Rous Sarcoma Virus [Gorman et al. (1982b) Proc. Natl. Acad. Sci. 79:6777] and from human cytomegalovirus [Boshart et al. (1985) Cell 41:521]. Additionally, some enhancers are regulatable and become active only in the presence of an inducer, such as a hormone or metal ion [Sassone-Corsi and Borelli (1986) Trends Genet. 2:215; Maniatis et al. (1987) Science 236:1237].

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus triparite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation [Birnstiel et al. (1985) Cell 41:349; Proudfoot and Whitelaw (1988) “Termination and 3′ end processing of eukaryotic RNA. In Transcription and splicing (ed. B. D. Hames and D. M. Glover); Proudfoot (1989) Trends Biochem. Sci. 14:105]. These sequences direct the transcription of an mRNA which can be translated into the polypeptide encoded by the DNA. Examples of transcription terminater/polyadenylation signals include those derived from SV40 [Sambrook et al (1989) “Expression of cloned genes in cultured mammalian cells.” In Molecular Cloning: A Laboratory Manual]. Usually, the above described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (e.g. plasmids) capable of stable maintenance in a host, such as mammalian cells or bacteria. Mammalian replication systems include those derived from animal viruses, which require trans-acting factors to replicate. For example, plasmids containing the replication systems of papovaviruses, such as SV40 [Gluzman (1981) Cell 23:175] or polyomavirus, replicate to extremely high copy number in the presence of the appropriate viral T antigen. Additional examples of mammalian replicons include those derived from bovine papillomavirus and Epstein-Barr virus. Additionally, the replicon may have two replication systems, thus allowing it to be maintained, for example, in mammalian cells for expression and in a prokaryotic host for cloning and amplification. Examples of such mammalian-bacteria shuttle vectors include pMT2 [Kaufman et al. (1989) Mol. Cell. Biol. 9:946] and pHEBO [Shimizu et al. (1986) Mol. Cell. Biol. 6:1074]. The transformation procedure used depends upon the host to be transformed. Methods for introduction of heterologous polynucleotides into mammalian cells are known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are known in the art and include many immortalized. cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g. Hep G2), and a number of other cell lines.

ii. Baculovirus Systems

The polynucleotide encoding the protein can also be inserted into a suitable insect expression vector, and is operably linked to the control elements within that vector. Vector construction employs techniques which are known in the art Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media.

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego Calif. (“MaxBac” kit). These techniques are generally known to those skilled in the art and fully described in Summers & Smith, Texas Agricultural Experiment Station Bulletin No. 1555 (1987) (“Summers & Smith”).

Prior to inserting the DNA sequence encoding the protein into the baculovirus genome, the above described components, comprising a promoter, leader (if desired), coding sequence, and transcription termination sequence, are usually assembled into an intermediate transplacement construct (transfer vector). This may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extra-chromosomal element (e.g. plasmids) capable of stable maintenance in a host, such as a bacterium. The replicon will have a replication system, thus allowing it to be maintained in a suitable host for cloning and amplification. Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT; see Luckow and Summers, Virology (1989) 17:31. The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Virol. 69:765.

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human α-interferon, Maeda et al., (1985), Nature 315:592; human gastrin-releasing peptide, Lebacq-Verheyden et al., (1988), Molec. Cell. Biol. 8:3129; human IL-2, Smith et al., (1985) Proc. Nat'l Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art. (See Summers & Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91. The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between about 1% and about 5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wild-type virus, the transfection supernatant is plaqued onto a monolayer of insect cells by techniques known to those skilled in the art. Namely, the plaques are screened under the light microscope for the presence (indicative of wild-type virus) or absence (indicative of recombinant virus) of occlusion bodies. “Current Protocols in Microbiology” Vol. 2 (Ausubel et al. eds) at 16.8 (Supp. 10, 1990); Summers & Smith, supra; Miller et al. (1989).

Recombinant baculovirus expression vectors have been developed for infection into several insect cells. For example, recombinant baculoviruses have been developed for, inter alia: Aedes aegypti, Autographa californica, Bombyx mori, Drosophila melanogaster, Spodoptera frugiperda, and Trichoplusia ni (WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Cells and cell culture media are commercially available for both direct and fusion expression of heterologous polypeptides in a baculovirus/expression system; cell culture technology is generally known to those skilled in the art. See, e.g. Summers & Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, e.g. HPLC, affinity chromatography, ion exchange chromatography, etc.; electrophoresis; density gradient centrifugation; solvent extraction, etc. As appropriate, the product may be further purified, as required, so as to remove substantially any insect proteins which are also present in the medium, so as to provide a product which is at least substantially free of host debris, e.g. proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iii. Plant Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture has been described by Zenk, Phytochemistry 30:3861-3863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMillin, Gibberellins: in: Advanced Plant Physiology, Malcolm B. Wilkins, ed., 1984 Pitman Publishing Limited, London, pp. 21-52. References that describe other metabolically-regulated genes: Sheen, Plant Cell, 2:1027-1038 (1990); Maas et al., EMBO J. 9:3447-3452 (1990); Benkel and Hickey, Proc. Natl. Acad. Sci. 84:1337-1339 (1987). Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilnink and Dons, 1993, Plant Mol. Biol. Reptr, 11(2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl. Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iv. Bacterial Systems

Bacterial expression techniques are known in the art. A bacterial promoter is any DNA sequence capable of binding bacterial RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) [Raibaud et al. (1984) Annu. Rev. Genet. 18:173]. Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) [Chang et al. (1977) Nature 198:1056], and maltose. Additional examples include promoter sequences derived from biosynthetic enzymes such as tryptophan (trp) [Goeddel et al. (1980) Nuc. Acids Res. 8:4057; Yelverton et al. (1981) Nucl. Acids Res. 9:731; U.S. Pat. No. 4,738,921; EP-A-0036776 and EP-A-0121775]. The g-laotamase (bla) promoter system [Weissmann (1981) “The cloning of interferon and other mistakes.” In Interferon 3 (ed. I. Gresser)], bacteriophage lambda PL [Shimatake et al. (1981) Nature 292:128] and T5 [U.S. Pat. No. 4,689,406] promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the tac promoter is a hybrid trp-lac promoter comprised of both trp promoter and lac operon sequences that is regulated by the lac repressor [Amann et al. (1983) Gene 25:167; de Boer et al. (1983) Proc. Natl. Acad. Sci. 80:21]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al. (1986) J. Mol. Biol. 189:113; Tabor et al. (1985) Proc Natl. Acad. Sci. 82:1074]. In addition, a hybrid promoter can also be comprised of a bacteriophage promoter and an E. coli operator region (EPO-A-0 267 851).

In addition to a functioning promoter sequence, an efficient ribosome binding site is also useful for the expression of foreign genes in prokaryotes. In E. coli, the ribosome binding site is called the Shine-Dalgarno (SD) sequence and includes an initiation codon (ATG) and a sequence 3-9 nucleotides in length located 3-11 nucleotides upstream of the initiation codon [Shine et al. (1975) Nature 254:34]. The SD sequence is thought to promote binding of mRNA to the ribosome by the pairing of bases between the SD sequence and the 3′ and of E. coli 16S rRNA [Steitz et al. (1979) “Genetic signals and nucleotide sequences in messenger RNA.” In Biological Regulation and Development: Gene Expression (ed. R. F. Goldberger)]. To express eukaryotic genes and prokaryotic genes with weak ribosome-binding site [Sambrook et al. (1989) “Expression of cloned genes in Escherichia coli.” In Molecular Cloning. A Laboratory Manual].

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo on in vitro incubation with a bacterial methionine N-terminal peptidase (EPO-A-0 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene [Nagai et al. (1984) Nature 309:810]. Fusion proteins can also be made with sequences from the lacZ [Jia et al. (1987) Gene 60:197], trpE [Allen et al. (1987) J. Biotechnol. 5:93; Makoff et al. (1989) J. Gen. Microbiol. 135:11], and Chey [EP-A-0 324 647] genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (e.g. ubiquitin specific processing-protease) to cleave the ubiquitin from the foreign protein. Through this method, native foreign protein can be isolated [Miller et al. (1989) Bio/Technology 7:698].

Alternatively, foreign proteins can also be secreted from the cell by creating chimeric DNA molecules that encode a fusion protein comprised of a signal peptide sequence fragment that provides for secretion of the foreign protein in bacteria [U.S. Pat. No. 4,336,336]. The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) [Masui et al. (1983), in: Experimental Manipulation of Gene Expression; Ghrayeb et al. (1984) EMBO J 3:2437] and the E. coli alkaline phosphatase signal sequence (phoA) [Oka et al. (1985) Proc. Natl. Acad. Sci. 82:7212]. As an additional example, the signal sequence of the alpha-amylase gene from various Bacillus strains can be used to secrete heterologous proteins from B. subtilis [Palva et al. (1982) Proc. Natl. Acad. Sci. USA 79:5582; EP-A-0 244 042].

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (e.g. plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous. DNA in the vector and the bacterial chromosome. For example, integrating vectors constructed with DNA from various Bacillus strains integrate into the Bacillus chromosome (EP-A-0 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline [Davies et al. (1978) Annu. Rev. Microbiol. 32:469]. Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis [Palva et al. (1982) Proc. Natl. Acad. Sci. USA 79:5582; EP-A-0 036 259 and EP-A-0 063 953; WO 84/04541], Escherichia coli [Shimatake et al. (1981) Nature 292:128; Amann et al. (1985) Gene 40:183; Studier et al. (1986) J. Mol. Biol. 189:113; EP-A-0 036 776, EP-A-0 136 829 and EP-A-0 136 907], Streptococcus cremoris [Powell et al. (1988) Appl. Environ. Microbiol. 54:655]; Streptococcus lividans [Powell et al. (1988) Appl. Environ. Microbiol. 54:655], Streptomyces lividans [U.S. Pat. No. 4,745,056].

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. See e.g. [Masson et al. (1989) FEMS Microbiol. Lett. 60:273; Palva et al. (1982) Proc. Natl. Acad. Sci. USA 79:5582; EP-A-0 036 259 and EP-A-0 063 953; WO 84/04541, Bacillus], [Miller et al. (1988) Proc. Natl. Acad. Sci. 85:856; Wang et al. (1990) J. Bacterol. 172:949, Campylobacter], [Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids. In Genetic Engineering: Proceedings of the International Symposium on Genetic Engineering (eds. H. W. Boyer and S, Nicosia); Mandel et al. (1970) J. Mol. Biol. 53:159; Taketo (1988) Biochim. Biophys. Acta 949:318; Escherichia], [Chassy et al. (1987) FEMS Microbiol. Lett. 44:173 Lactobacillus]; [Fiedler et al. (1988) Anal. Biochem 170:38, Pseudomonas]; [Augustin et al. (1990) FEMS Microbiol. Lett. 66:203, Staphylococcus], [Barany et al. (1980) J. Bacteriol. 144:698; Harlander (1987) “Transformation of Streptococcus lactis by electroporation, in: Streptococcal Genetics (ed. J. Ferretti and R. Curtiss III); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412, Streptococcus].

v. Yeast Expression

Yeast expression systems are also known to one of ordinary skill in the art. A yeast promoter is any DNA sequence capable of binding yeast RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EP-A-0 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO-A-0 329 203). The yeast PHO5 gene, encoding acid phosphatase, also provides useful promoter sequences [Myanohara et al. (1983) Proc. Natl. Acad. Sci. USA 80:1].

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK (EP-A-0 164 556). Furthermore, a yeast promoter can include naturally occurring promoters of non-yeast origin that have the ability to bind yeast RNA polymerase and initiate transcription. Examples of such promoters include, inter alia, [Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11:163; Panthier et al. (1980) Curr. Genet. 2:109;].

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See e.g. EP-A-0 196 056. Another example is a ubiquitin fusion protein. Such a fusion protein is made with the ubiquitin region that preferably retains a site for a processing enzyme (e.g. ubiquitin-specific processing protease) to cleave the ubiquitin from the foreign protein. Through this method, therefore, native foreign protein can be isolated (e.g. WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the yeast invertase gene (EP-A-0 012 873; JPO. 62,096,086) and the A-factor gene (U.S. Pat. No. 4,588,684). Alternatively, leaders of non-yeast origin, such as an interferon leader, exist that also provide for secretion in yeast (EP-A-0 060 057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EP-A-0 324 274). Additional leaders employing an alpha-factor leader fragment that provides for secretion include hybrid alpha-factor leaders made with a presequence of a first yeast, but a pro-region from a second yeast alphafactor. (e.g. see WO 89/02463.)

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (e.g. plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 [Botstein et al. (1979) Gene 8:17-24], pCl/1 [Brake et al. (1984) Proc. Natl. Acad. Sci. USA 81:4642-4646], and YRp17 [Stinchcomb et al. (1982) J. Mol. Biol. 158:157]. In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See e.g. Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome [Orr-Weaver et al. (1983) Methods in Enzymol. 101:228-245]. An integrating vector may be directed to a specific locus in yeast by selecting the appropriate homologous sequence for inclusion in the vector. See Orr-Weaver et al., supra. One or more expression construct may integrate, possibly affecting levels of recombinant protein produced [Rine et al. (1983) Proc. Natl. Acad. Sci. USA 80:6750]. The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions [Butt et al. (1987) Microbiol, Rev. 51:351].

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors have been developed for, inter alia, the following yeasts: Candida albicans [Kurtz, et al. (1986) Mol. Cell. Biol. 6:142], Candida maltosa [Kunze, et al. (1985) J. Basic Microbiol. 25:141]. Hansenula polymorpha [Gleeson, et al. (1986) J. Gen. Microbiol. 132:3459; Roggenkamp et al. (1986) Mol. Gen. Genet. 202:302], Kluyveromyces fragilis [Das, et al. (1984) J. Bacteriol. 158:1165], Kluyveromyces lactis [De Louvencourt et al. (1983) J. Bacteriol. 154:737; Van den Berg et al. (1990) Bio/Technology 8:135], Pichia guillerimondii [Kunze et al. (1985) J. Basic Microbiol. 25:141], Pichia pastoris [Cregg, et al. (1985) Mol. Cell. Biol. 5:3376; U.S. Pat. Nos. 4,837,148 and 4,929,555], Saccharomyces cerevisiae [linnen et al. (1978) Proc. Natl. Acad. Sci. USA 75:1929; Ito et al. (1983) J. Bacteriol. 153:163], Schizosaccharomyces pombe [Beach and Nurse (1981) Nature 300:706], and Yarrowia lipolytica [Davidow, et al. (1985) Curr. Genet. 10:380471 Gaillardin, et al. (1985) Curr. Genet. 10:49].

Methods of introducing exogenous DNA into yeast hosts are well-known in the art, and usually include either the transformation of spheroplasts or of intact yeast cells treated with alkali cations. Transformation procedures usually vary with the yeast species to be transformed. See e.g. [Kurtz et al. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (194) J. Bacteriol. 158:1165; De Louvencourt et al. (1983) J. Bacteriol. 154:1165; Van den Berg et al. (1990) BioTechnology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 and 4,929,555; Pichia]; [Hinnen et al. (1978) Proc. Natl. Acad. Sci. USA 75; 1929; Ito et al. (1983) J. Bacteriol. 153:163 Saccharomyces]; [Beach and Nurse (1981) Nature 300:706; Schizosaccharomyces]; [Davidow et al. (1985) Curr. Genet. 10:39; Gaillardin et al. (1985) Curr. Genet. 10:49; Yarrowia].

### Antibodies

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

Antibodies against the proteins of the invention are useful for affinity chromatography, immunoassays, and distinguishing/identifying Streptococcal proteins.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antisera is obtained by bleeding the immunized animal into a glass or plastic container, incubating the blood at 25° C. for one hour, followed by incubating at 4° C. for 2-18 hours. The serum is recovered by centrifugation (e.g. 1,000 g for 10 minutes). About 20-50 ml per bleed may be obtained from rabbits.

Monoclonal antibodies are prepared using the standard method of Kohler & Milstein [Nature (1975) 256:495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (e.g. hypoxanthine, aminopterin, thymidine medium, “HAT”). The resulting hybridomas are plated by limiting dilution, and are assayed for production of antibodies which bind specifically to the immunizing antigen (and which do not bind to unrelated antigens). The selected MAb-secreting hybridomas are then cultured either in vitro (e.g. in tissue culture bottles or hollow fiber reactors), or in vivo (as ascites in mice). If desired, the antibodies (whether polyclonal or monoclonal) may be labeled using conventional techniques. Suitable labels include fluorophores, chromophores, radioactive atoms (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the instant invention.

### Pharmaceutical Compositions

Pharmaceutical compositions can comprise either polypeptides, antibodies, or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991). Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

### Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g. see WO98/20734), needles, and gene guns or hyposprays. Dosage treatment may be a single dose schedule or a multiple dose schedule.

See also Delivery Strategies for Antisense Oligonucleotide Therapeutics (ed. Akhtar) ISBN 0849347785.

### Vaccines

Vaccines according to the invention may either be prophylactic (i.e. to prevent infection) or therapeutic (i.e. to treat disease after infection).

Such vaccines comprise immunising antigen(s), immunogen(s), polypeptide(s), protein(s) or nucleic acid, usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogens.

Vaccines of the invention may be administered in conjunction with other immunoregulatory agents. In particular, compositions will usually include an adjuvant.

Preferred further adjuvants include, but are not limited to, one or more of the following set forth below:

### A. Mineral Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphoshpates, orthophosphates), sulphates, etc. {e.g. see chapters 8 & 9 of ref. 1}), or mixtures of different mineral compounds, with the compounds taking any suitable form (e.g. gel, crystalline, amorphous, etc.), and with adsorption being preferred. The mineral containing compositions may also be formulated as a particle of metal salt. See ref. 2.

### B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See ref. 3.

Complete Freund's adjuvant (CFA) and incomplete Freund's adjuvant (IFA) may also be used as adjuvants in the invention.

### C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO 96/33739).

Combinations of saponins and cholesterols can be used to form unique particles called Immunostimulating Complexs (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP 0 109 942, WO 96/11711 and WO 96/33739. Optionally, the ISCOMS may be devoid of additional detergent. See ref. 4.

A review of the development of saponin based adjuvants can be found at ref. 5.

### C. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO 03/024480, WO 03/024481, and Refs. 6, 7, 8 and 9. Virosomes are discussed further in, for example, Ref. 10

### D. Bacterial or Microbial Derivatives

Adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Ref. 11.

(2) Lipid A Derivatives

Lipid A derivatives include derivatives of lipid A from Escherichia coli such as OM-174. OM-174 is described for example in Ref. 12 and 13.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See ref. 14, WO 02/26757 and WO 99/62923 for examples of possible analog substitutions. The adjuvant effect of CpG oligonucleotides is further discussed in Refs. 15, 16, WO 98/40100, U.S. Pat. No. 6,207,646, U.S. Pat. No. 6,239,116, and U.S. Pat. No. 6,429,199.

The CpG sequence may be directed to TLR9, such as the motif GTCGTT or ITCGTT. See ref. 17. The CpG sequence may be specific for inducing a Th1 immune response, such as a CpG-A ODN, or it may be more specific for inducing a B cell response, such a CpG-B ODN. CpG-A and CpG-B ODNs are discussed in refs. 18, 19 and WO 01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 20, 21, 22 and WO 03/035836.

(4) ADP-Ribosylating Toxins and Detoxified Derivatives Thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO 95/17211 and as parenteral adjuvants in WO 98/42375. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in Refs. 23, 24, 25, 26, 27, 28, 29 and 30 each of which is specifically incorporated by reference herein in their entirety. Numerical reference for amino acid substitutions is preferably based on the alignments of the A and B subunits of ADP-ribosylating toxins set forth in Domenighini et al., Mol. Microbiol (1995) 15(6):1165-1167, specifically incorporated herein by reference in its entirety.

### E. Human Immunomodulators

Human immunomodulators suitable for use as adjuvants in the invention include cytokines, such as interleukins (e.g. II-1, IL-2, IL-4, IL-5, IL-6, IL-7, IL-12, etc.), interferons (e.g. interferon-?), macrophage colony stimulating factor, and tumor necrosis factor.

### F. Bioadhesives and Mucoadhesives

Bioadhesives and mucoadhesives may also be used as adjuvants in the invention. Suitable bioadhesives include esterified hyaluronic acid microspheres (Ref 31) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g., ref. 32.

### G. Microparticles

Microparticles may also be used as adjuvants in the invention. Microparticles (i.e. a particle of ˜100nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(a-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### H. Liposomes

Examples of liposome formulations suitable for use as adjuvants are described in U.S. Pat. No. 6,090,406, U.S. Pat. No. 5,916,588, and EP 0 626 169.

I. Polyoxyethylene ether and Polyoxyethylene Ester Formulations

Adjuvants suitable for use in the invention include polyoxyethylene ethers and polyoxyethylene esters. Ref. 33. Such formulations further include polyoxyethylene sorbitan ester surfactants in combination with an octoxynol (Ref. 34) as well as polyoxyethylene alkyl ethers or ester surfactants in combination with at least one additional non-ionic surfactant such as an octoxynol (Ref. 35).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### J. Polyphosphazene (PCPP)

PCPP formulations are described, for example, in Ref 36 and 37.

### K. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### L. Imidazoguinolone Compounds.

Examples of imidazoquinolone compounds suitable for use adjuvants in the invention include Imiquamod and its homologues, described further in Ref. 38 and 39.

The invention may also comprise combinations of aspects of one or more of the adjuvants identified above. For example, the following adjuvant compositions may be used in the invention:

(1) a saponin and an oil-in-water emulsion (ref. 40);

(2) a saponin (e.g., QS21)+a non-toxic LPS derivative (e.g., 3dMPL) (see WO 94/00153);

(3) a saponin (e.g., QS21)+a non-toxic LPS derivative (e.g., 3dMPL)+a cholesterol;

(4) a saponin (e.g. QS21)+3dMPL+IL-12 (optionally+a sterol) (Ref. 41); combinations of 3dMPL with, for example, QS21 and/or oil-in-water emulsions (Ref. 42);

(5) SAF, containing 10% Squalane, 0.4% Tween 80, 5% pluronic-block polymer L121, and thr-MDP, either microfluidized into a submicron emulsion or vortexed to generate a larger particle size emulsion.

(6) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (DM), and cell wall skeleton (CWS), preferably MPL+CWS (Detox™); and

(7) one or more mineral salts (such as an aluminum salt)+a non-toxic derivative of LPS (such as 3dPML).

Aluminium salts and MF59 are preferred adjuvants for parenteral immunisation. Mutant bacterial toxins are preferred mucosal adjuvants.

The immunogenic compositions (e.g. the immunising antigen/immunogen/polypeptide/protein/nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (e.g. nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The immunogenic compositions are conventionally administered parenterally, e.g. by injection, either subcutaneously, intramuscularly, or transdermally/transcutaneously (e.g. WO98/20734). Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

As an alternative to protein-based vaccines, DNA vaccination may be used [e.g. Robinson & Torres (1997) Seminars in Immunol 9:271-283; Donnelly et al. (1997) Annu Rev Immunol 15:617-648; later herein].

### Gene Delivery Vehicles

Gene therapy vehicles for delivery of constructs including a coding sequence of a therapeutic of the invention, to be delivered to the mammal for expression in the mammal, can be administered either locally or systemically. These constructs can utilize viral or non-viral vector approaches in in vivo or ex vivo modality. Expression of such coding sequence can be induced using endogenous mammalian or heterologous promoters. Expression of the coding sequence in vivo can be either constitutive or regulated. The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-64; Kimura (1994) Human Gene Therapy 5:845-852; Connelly (1995) Human Gene Therapy 6:185-193; and Kaplitt (1994) Nature Genetics 6:148-153.

Retroviral vectors are well known in the art and we contemplate that any retroviral gene therapy vector is employable in the invention, including B, C and D type retroviruses, xenotropic retroviruses (for example, NZB-X1, NZB-X2 and NZB9-1 (see ONeill (1985) J. Virol. 53:160) polytropic retroviruses e.g. MCF and MCF-MLV (see Kelly (1983) J. Virol. 45:291), spumaviruses and lentiviruses. See RNA Tumor Viruses, Second Edition, Cold Spring Harbor Laboratory, 1985.

Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Pat. No. 5,591,624). Retrovirus vectors can be constructed for site-specific integration into host cell DNA by incorporation of a chimeric integrase enzyme into the retroviral particle (see WO96/37626). It is preferable that the recombinant viral vector is a replication defective recombinant virus.

Packaging cell lines suitable for use with the above-described retrovirus vectors are well known in the art, are readily prepared (see WO95/30763 and WO92/05266), and can be used to create producer cell lines (also termed vector cell lines or “VCLs”) for the production of recombinant vector particles. Preferably, the packaging cell lines are made from human parent cells (e.g. HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J Virol 19:19-25), Abelson (ATCC No. VR-999), Friend (ATCC No. VR-245), Graffi, Gross (ATCC Nol VR-590), Kirsten, Harvey Sarcoma Virus and Rauscher (ATCC No. VR-998) and MQloney Murine Leukemia Virus (ATCC No. VR-190). Such retroviruses may be obtained from depositories or collections such as the American Type Culture Collection (“ATCC”) in Rockville, Md. or isolated from known sources using commonly available techniques. Exemplary known retroviral gene therapy vectors employable in this invention include those described in patent applications GB2200651, EP0415731, EP0345242, EP0334301, WO89/02468; WO89/05349, WO89/09271, WO90/02806, WO90/07936, WO94/03622, WO93/25698, WO93/25234, WO93/11230, WO93/10218, WO91/02805, WO91/02825, WO95/07994, U.S. Pat. No. 5,219,740, U.S. Pat. No. 4,405,712, U.S. Pat. No. 4,861,719, U.S. Pat. No. 4,980,289, U.S. Pat. No. 4,777,127, U.S. Pat. No. 5,591,624. See also Vile (1993) Cancer Res 53:3860-3864; Vile (1993) Cancer Res 53:962-967; Ram (1993) Cancer Res 53 (1993) 83-88; Takamiya (1992) J Neurosci Res 33:493-503; Baba (1993) J Neurosurg 79:729-735; Mann (1983) Cell 33:153; Cane (1984) Proc Natl Acad Sci 81:6349; and Miller (1990) Human Gene Therapy 1.

Human adenoviral gene therapy vectors are also known in the art and employable in this invention. See, for example, Berkner (1988) Biotechniques 6:616 and Rosenfeld (1991) Science 252:431, and WO93/07283, WO93/06223, and WO93/07282. Exemplary known adenoviral gene therapy vectors employable in this invention include those described in the above referenced documents and in WO94/12649, WO93/03769, WO93/19191, WO94/28938, WO95/11984, WO95/00655, WO95/27071, WO95/29993, WO95/34671, WO96/05320, WO94/08026, WO94/11506, WO93/06223, WO94/24299, WO95/14102, WO95/24297, WO95/02697, WO94/28152, WO94/24299, WO95/09241, WO95/25807, WO95/05835, WO94/18922 and WO95/09654. Alternatively, administration of DNA linked to killed adenovirus as described in Curiel (1992) Hum. Gene Ther. 3:147-154 may be employed. The gene delivery vehicles of the invention also include adenovirus associated virus (AAV) vectors. Leading and preferred examples of such vectors for use in this invention are the AAV-2 based vectors disclosed in Srivastava, WO93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (i.e. there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini (1993) Gene 124:257-262. Another example of such an AAV vector is psub201 (see Samulski (1987) J. Virol. 61:3096). Another exemplary AAV vector is the Double-D ITR vector. Construction of the Double-D ITR vector is disclosed in U.S. Pat. No. 5,478,745. Still other vectors are those disclosed in Carter U.S. Pat. No. 4,797,368 and Muzyczka U.S. Pat. No. 5,139,941, Chartejee U.S. Pat. No. 5,474,935, and Kotin WO94/288157. Yet a further example of an AAV vector employable in this invention is SSV9AFABTKneo, which contains the AFP enhancer and albumin promoter and directs expression predominantly in the liver. Its structure and construction are disclosed in Su (1996) Human Gene Therapy 7:463-470. Additional AAV gene therapy vectors are described in U.S. Pat. No. 5,354,678, U.S. Pat. No. 5,173,414, U.S. Pat. No. 5,139,941, and U.S. Pat. No. 5,252,479.

The gene therapy vectors of the invention also include herpes vectors. Leading and preferred examples are herpes simplex virus vectors containing a sequence encoding a thymidine kinase polypeptide such as those disclosed in U.S. Pat. No. 5,288,641 and EP0176170 (Roizman). Additional exemplary herpes simplex virus vectors include HFEM/ICP6-LacZ disclosed in WO95/04139 (Wistar Institute), pHSVlac described in Geller (1988) Science 241:1667-1669 and in WO90/09441 and WO92/07945, HSV Us3::pgC-lacZ described in Fink (1992) Human Gene Therapy 3:11-19 and HSV 7134, 2 RH 105 and GAL4 described in EP 0453242 (Breakefield), and those deposited with the ATCC with accession numbers VR-977 and VR-260.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO92/10578. More particularly, those alpha virus vectors described in U.S. Ser. No. 08/405,627, filed Mar. 15, 1995, WO94/21792, WO92/10578, WO95/07994, U.S. Pat. No. 5,091,309 and U.S. Pat. No. 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see U.S. Ser. No. 08/679,640).

DNA vector systems such as eukaryotic layered expression systems are also useful for expressing the nucleic acids of the invention. See WO95/07994 for a detailed description of eukaryotic layered expression systems. Preferably, the eukaryotic layered expression systems of the invention are derived from alphavirus vectors and most preferably from Sindbis viral vectors.

Other viral vectors suitable for use in the present invention include those derived from poliovirus, for example ATCC VR-58 and those described in Evans, Nature 339 (1989) 385 and Sabin (1973) J. Biol. Standardization 1:115; rhinovirus, for example ATCC VR-1110 and those described in Arnold (1990) J Cell Biochem L401; pox viruses such as canary pox virus or vaccinia virus, for example ATCC VR-111 and ATCC VR-2010 and those described in Fisher-Hoch (1989) Proc Natl Acad Sci 86:317; Flexner (1989) Ann NY Acad Sci 569:86, Flexner (1990) Vaccine 8:17; in U.S. Pat. No. 4,603,112 and U.S. Pat. No. 4,769,330 and WO89/01973; SV40 virus, for example ATCC VR-305 and those described in Mulligan (1979) Nature 277:108 and Madzak (1992) J Gen Virol 73:1533; influenza virus, for example ATCC VR-797 and recombinant influenza viruses made employing reverse genetics techniques as described in U.S. Pat. No. 5,166,057 and in Enami (1990) Proc Natl Acad Sci 87:3802-3805; Enami & Palese (1991) J Virol 65:2711-2713 and Luytjes (1989) Cell 59:110, (see also McMichael (1983) NEJ Med 309:13, and Yap (1978) Nature 273:238 and Nature (1979) 277:108); human immunodeficiency virus as described in EP-0386882 and in Buchschacher (1992) J. Virol. 66:2731; measles virus, for example ATCC VR-67 and VR-1247 and those described in EP-0440219; Aura virus, for example ATCC VR-368; Bebaru virus, for example ATCC VR-600 and ATCC VR-1240; Cabassou virus, for example ATCC VR-922; Chikungunya virus, for example ATCC VR-64 and ATCC VR-1241; Fort Morgan Virus, for example ATCC VR-924; Getah virus, for example ATCC VR-369 and ATCC VR-1243; Kyzylagach virus, for example ATCC VR-927; Mayaro virus, for example ATCC VR-66; Mucambo virus, for example ATCC VR-580 and ATCC VR-1244; Ndumu virus, for example ATCC VR-371; Pixuna virus, for example ATCC VR-372 and ATCC VR-1245; Tonate virus, for example ATCC VR-925; Triniti virus, for example ATCC VR-469; Una virus, for example ATCC VR-374; Whataroa virus, for example ATCC VR-926; Y-62-33 virus, for example ATCC VR-375; O'Nyong virus, Eastern encephalitis virus, for example ATCC VR-65 and ATCC VR-1242; Western encephalitis virus, for example ATCC VR-70, ATCC VR-1251, ATCC VR-622 and ATCC VR-1252; and coronavirus, for example ATCC VR-740 and those described in Hamre (1966) Proc Soc Exp Biol Med 121:190.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994 and Curiel (1992) Hum Gene Ther 3:147-154 ligand linked DNA, for example see Wu (1989) J Biol Chem 264:16985-16987, eucaryotic cell delivery vehicles cells, for example see U.S. Ser. No. 08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in WO92/11033, nucleic charge neutralization or fusion with cell membranes. Additional approaches are described in Philip (1994) Mol Cell Biol 14:2411-2418 and in Woffendin (1994) Proc Natl Acad Sci 91:1581-1585.

Particle mediated gene transfer may be employed, for example see U.S. Ser. No. 60/023,867. Briefly, the sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO95/13796, WO94/23697, WO91/14445 and EP-524,968. As described in U.S. Ser. No. 60/023,867, on non-viral delivery, the nucleic acid sequences encoding a polypeptide can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO92/11033

Exemplary liposome and polycationic gene delivery vehicles are those described in U.S. Pat. Nos. 5,422,120 and 4,762,915; in WO 95/13796; WO94/23697; and WO91/14445; in EP-0524968; and in Stryer, Biochemistry, pages 236-240 (1975) W.H. Freeman, San Francisco; Szoka (1980) Biochem Biophys Acta 600:1; Bayer (1979) Biochem Biophys Acta 550:464; Rivnay (1987) Meth Enzymol 149:119; Wang (1987) Proc Natl Acad Sci 84:7851; Plant (1989) Anal Biochem 176:420.

A polynucleotide composition can comprises therapeutically effective amount of a gene therapy vehicle, as the term is defined above. For purposes of the present invention, an effective dose will be from about 0.01 mg/kg to 50 mg/kg or 0.05 mg/kg to about 10 mg/kg of the DNA constructs in the individual to which it is administered.

### Delivery Methods

Once formulated, the polynucleotide compositions of the invention can be administered (1) directly to the subject; (2) delivered ex vivo, to cells derived from the subject; or (3) in vitro for expression of recombinant proteins. The subjects to be treated can be mammals or birds. Also, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal or transcutaneous applications (e.g. see WO98/20734), needles, and gene guns or hyposprays. Dosage treatment may be a single dose schedule or a multiple dose schedule.

Methods for the ex vivo delivery and reimplantation of transformed cells into a subject are known in the art and described in e.g. WO93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

### Polynucleotide and Polypeptide Pharmaceutical Compositions

The terms “polynucleotide” and “nucleic acid”, used interchangeably herein,

In addition to the pharmaceutically acceptable carriers and salts described above, the following additional agents can be used with polynucleotide and/or polypeptide compositions.

### A. Polypeptides

One example are polypeptides which include, without limitation: asioloorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

### B. Hormones, Vitamins, etc.

Other groups that can be included are, for example: hormones, steroids, androgens, estrogens, thyroid hormone, or vitamins, folic acid.

### C. Polyalkylenes, Polysaccharides, etc.

Also, polyalkylene glycol can be included with the desired polynucleotides/polypeptides. In a preferred embodiment, the polyalkylene glycol is polyethlylene glycol. In addition, mono-, di-, or polysaccharides can be included. In a preferred embodiment of this aspect, the polysaccharide is dextran or DEAE-dextran. Also, chitosan and poly(lactide-co-glycolide)

### D. Lipids, and Liposomes

The desired polynucleotide/polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Felgner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Felgner supra). Other commercially available liposomes include transfectace (DDAB/DOPE) and DOTAP/DOPE (Boerhinger). Other cationic liposomes can be prepared from readily available materials using techniques well known in the art. See, e.g. Szoka (1978) Proc. Natl. Acad. Sci. USA 75:4194-4198; WO90/11092 for a description of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See e.g. Straubinger (1983) Meth. Immunol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

### E. Lipoproteins

In addition, lipoproteins can be included with the polynucleotide/polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein. For example, naturally occurring chylomicrons comprises of A, B, C & E, over time these lipoproteins lose A and acquire C & E. VLDL comprises A, B, C & E apoproteins, LDL comprises apoprotein B; and HDL comprises apoproteins A, C, & E.

The amino acid of these apoproteins are known and are described in, for example, Breslow (1985) Annu Rev. Biochem 54:699; Law (1986) Adv. Exp Med. Biol. 151:162; Chen (1986) J Biol Chem 261:12918; Kane (1980) Proc Natl Acad Sci USA 77:2465; and Utermann (1984) Hum Genet. 65:232.

Lipoproteins contain a variety of lipids including, triglycerides, cholesterol (free and esters), and phospholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mahey (1979) J. Clin. Invest 64:743-750. Lipoproteins can also be produced by in vitro or recombinant methods by expression of the apoprotein genes in a desired host cell. See, for example, Atkinson (1986) Annu Rev Biophys Chem 15:403 and Radding (1958) Biochim Biophys Acta 30: 443. Lipoproteins can also be purchased from commercial suppliers, such as Biomedical Techniologies, Inc., Stoughton, Mass., USA. Further description of lipoproteins can be found in Zuckermann et al. PCT/US97/14465.

### F. Polycationic Agents

Polycationic agents can be included, with or without lipoprotein, in a composition with the desired polynucleotide/polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples include histones, protamines, human serum albumin, DNA binding proteins, non-histone chromosomal proteins, coat proteins from DNA viruses, such as (×174, transcriptional factors also contain domains that bind DNA and therefore may be useful as nucleic aid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spermine, spermidine, and purtrescine.

The dimensions and of the physical properties of a polycationic agent can be extrapolated from the list above, to construct other polypeptide polycationic agents or to produce synthetic polycationic agents.

Synthetic polycationic agents which are useful include, for example, DEAE-dextran, polybrene. Lipofectin™, and lipofectAMINE™ are monomers that form polycationic complexes when combined with polynucleotides/polypeptides.

### Immunodiagnostic Assays

Streptococcus antigens of the invention can be used in immunoassays to detect antibody levels (or, conversely, anti-Streptococcus antibodies can be used to detect antigen levels). Immunoassays based on well defined, recombinant antigens can be developed to replace invasive diagnostics methods. Antibodies to Streptococcus proteins within biological samples, including for example, blood or serum samples, can be detected. Design of the immunoassays is subject to a great deal of variation, and a variety of these are known in the art. Protocols for the immunoassay may be based, for example, upon competition, or direct reaction, or sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Kits suitable for immunodiagnosis and containing the appropriate labeled reagents are constructed by packaging the appropriate materials, including the compositions of the invention, in suitable containers, along with the remaining reagents and materials (for example, suitable buffers, salt solutions, etc.) required for the conduct of the assay, as well as suitable set of assay instructions.

### Use of Polypeptides to Screen for Peptide Analogs and Antagonists

Polypeptides encoded by the instant polynucleotides and corresponding full length genes can be used to screen peptide libraries to identify binding partners, such as receptors, from within the library. Peptide libraries can be synthesized according to methods known in the art (e.g. U.S. Pat. No. 5,010,175; WO91/17823). Agonists or antagonists of the polypeptides if the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of a polypeptide binding partner, such as a receptor, encoded by a gene or a cDNA corresponding to a polynucleotide described herein, and at least one peptide agonist or antagonist of the binding partner. Such agonists and antagonists can be used to modulate, enhance, or inhibit receptor function in cells to which the receptor is native, or in cells that possess the receptor as a result of genetic engineering. Further, if the receptor shares biologically important characteristics with a known receptor, information about agonist/antagonist binding can facilitate development of improved agonists/antagonists of the known receptor.

### Identification of Anti-Bacterial Agents

### Drug Screening Assays

Of particular interest in the present invention is the identification of agents that have activity in modulating expression of one or more of the adhesion-specific genes described herein, so as to inhibit infection and/or disease. Of particular interest are screening assays for agents that have a low toxicity for human cells.

The term “agent” as used herein describes any molecule with the capability of altering or mimicking the expression or physiological function of a gene product of a differentially expressed gene. Generally a plurality of assay mixtures are run in parallel with different agent concentrations to obtain a differential response to the various concentrations. Typically, one of these concentrations serves as a negative control i.e. at zero concentration or below the level of detection.

Candidate agents encompass numerous chemical classes, including, but not limited to, organic molecules (e.g. small organic compounds having a molecular weight of more than 50 and less than about 2,500 daltons), peptides, antisense polynucleotides, and ribozymes, and the like. Candidate agents can comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: polynucleotides, peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

### Screening of Candidate Agents In Vitro

A wide variety of in vitro assays may be used to screen candidate agents for the desired biological activity, including, but not limited to, labeled in vitro protein-protein binding assays, protein-DNA binding assays (e.g. to identify agents that affect expression), electrophoretic mobility shift assays, immunoassays for protein binding, and the like. For example, by providing for the production of large amounts of a differentially expressed polypeptide, one can identify ligands or substrates that bind to, modulate or mimic the action of the polypeptide. The purified polypeptide may also be used for determination of three-dimensional crystal structure, which can be used for modeling intermolecular interactions, transcriptional regulation, etc.

The screening assay can be a binding assay, wherein one or more of the molecules may be joined to a label, and the label directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assays described herein. Where the assay is a binding assay, these include reagents like salts, neutral proteins, e.g. albumin, detergents, etc. that are used to facilitate optimal protein-protein binding, protein-DNA binding, and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Many mammalian genes have homologs in yeast and lower animals. The study of such homologs' physiological role and interactions with other proteins in vivo or in vitro can facilitate understanding of biological function. In addition to model systems based on genetic complementation, yeast has been shown to be a powerful tool for studying protein-protein interactions through the two hybrid system.

### Nucleic Acid Hybridisation

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al. [supra] Volume 2, chapter 9, pages 9.47 to 9.57.

“Stringency” refers to conditions in a hybridization reaction that favor association of very similar sequences over sequences that differ. For example, the combination of temperature and salt concentration should be chosen that is approximately 120 to 200° C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook et al. at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 μg for a plasmid or phage digest to 10⁻⁹ to 10⁻⁸ g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy mammalian gene a conservative approach would start with 10 μg of DNA, blot overnight, and hybridize overnight in the presence of 10% dextran sulfate using a probe of greater than 10⁸ cpm/μg, resulting in an exposure time of ˜24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:

Tm=81+16.6(log₁₀ Ci)+0.4[% (G+C)]−0.6(% formamide)−600/n−1.5(% mismatch).

where Ci is the salt concentration (monovalent ions) and n is the length of the hybrid in base pairs (slightly modified from Meinkoth & Wahl (1984) Anal. Biochem. 138: 267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (i.e. stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and reexposed. If the time required for exposure makes this approach impractical, several hybridization and/or washing stringencies should be tested in parallel.

### Nucleic Acid Probe Assays

Methods such as PCR, branched DNA probe assays, or blotting techniques utilizing nucleic acid probes according to the invention can determine the presence of cDNA or mRNA. A probe is said to “hybridize” with a sequence of the invention if it can form a duplex or double stranded complex, which is stable enough to be detected.

The nucleic acid probes will hybridize to the Streptococcus nucleotide sequences of the invention (including both sense and antisense strands). Though many different nucleotide sequences will encode the amino acid sequence, the native Streptococcal sequence is preferred because it is the actual sequence present in cells. mRNA represents a coding sequence and so a probe should be complementary to the coding sequence; single-stranded cDNA is complementary to mRNA, and so a cDNA probe should be complementary to the non-coding sequence.

The probe sequence need not be identical to the Streptococcal sequence (or its complement)-some variation in the sequence and length can lead to increased assay sensitivity if the nucleic acid probe can form a duplex with target nucleotides, which can be detected. Also, the nucleic acid probe can include additional nucleotides to stabilize the formed duplex. Additional Streptococcus sequence may also be helpful as a label to detect the formed duplex. For example, a non-complementary nucleotide sequence may be attached to the 5′ end of the probe, with the remainder of the probe sequence being complementary to a Streptococcus sequence. Alternatively, non-complementary bases or longer sequences can be interspersed into the probe, provided that the probe sequence has sufficient complementarity with the a Streptococcus sequence in order to hybridize therewith and thereby form a duplex which can be detected.

The exact length and sequence of the probe will depend on the hybridization conditions (e.g. temperature, salt condition etc.). For example, for diagnostic applications, depending on the complexity of the analyte sequence, the nucleic acid probe typically contains at least 10-20 nucleotides, preferably 15-25, and more preferably at least 30 nucleotides, although it may be shorter than this. Short primers generally require cooler temperatures to form sufficiently stable hybrid complexes with the template.

Probes may be produced by synthetic procedures, such as the triester method of Matteucci et al. [J. Am. Chem. Soc. (1981) 103:3185], or according to Urdea et al. [Proc. Natl. Acad. Sci. USA (1983) 80: 7461], or using commercially available automated oligonucleotide synthesizers.

The chemical nature of the probe can be selected according to preference. For certain applications, DNA or RNA are appropriate. For other applications, modifications may be incorporated e.g. backbone modifications, such as phosphorothioates or methylphosphonates, can be used to increase in vivo half-life, alter RNA affinity, increase nuclease resistance etc. [e.g. see Agrawal & Iyer (1995) Curr Opin Biotechnol 6:12-19; Agrawal (1996) TIBTECH 14:376-387]; analogues such as peptide nucleic acids may also be used [e.g. see Corey (1997) TIBTECH 15:224-229; Buchardt et al. (1993) TIBTECH 11:384-386].

Alternatively, the polymerase chain reaction (PCR) is another well-known means for detecting small amounts of target nucleic acid. The assay is described in Mullis et al. [Meth. Enzymol. (1987) 155:335-350] & U.S. Pat. Nos. 4,683,195 & 4,683,202. Two “primer” nucleotides hybridize with the target nucleic acids and are used to prime the reaction. The primers can comprise sequence that does not hybridize to the sequence of the amplification target (or its complement) to aid with duplex stability or, for example, to incorporate a convenient restriction site. Typically, such sequence will flank the desired Streptococcus sequence.

A thermostable polymerase creates copies of target nucleic acids from the primers using the original target nucleic acids as a template. After a threshold amount of target nucleic acids are generated by the polymerase, they can be detected by more traditional methods, such as Southern blots. When using the Southern blot method, the labelled probe will hybridize to the Streptococcus sequence (or its complement).

Also, mRNA or cDNA can be detected by traditional blotting techniques described in Sambrook et al [supra]. mRNA, or cDNA generated from mRNA using a polymerase enzyme, can be purified and separated using gel electrophoresis. The nucleic acids on the gel are then blotted onto a solid support, such as nitrocellulose. The solid support is exposed to a labelled probe and then washed to remove any unhybridized probe. Next, the duplexes containing the labeled probe are detected. Typically, the probe is labelled with a radioactive moiety.

### REFERENCES

1. Vaccine design: the subunit and adjuvant approach (1995) Powell & Newman. ISBN 0-306-44867-X.
2. WO00/23105.
3. WO90/14837.
4. WO00/07621.
5. Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.
6. Niikura et al., “Chimeric Recombinant Hepatitis E Virus-Like Particles as an Oral Vaccine Vehicle Presenting Foreign Epitopes”, Virology (2002) 293:273-280.
7. Lenz et al., “Papillomarivurs-Like Particles Induce Acute Activation of Dendritic Cells”, Journal of Immunology (2001) 5246-5355.
8. Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338.
9. Gerber et al., “Human Papillomavrisu Virus-Like Particles Are Efficient Oral Immunogens when Coadministered with Escherichia coli Heat-Labile Entertoxin Mutant R192G or CpG”, Journal of Virology (2001) 75(10):4752-4760.
10. Gluck et al., “New Technology Platforms in the Development of Vaccines for the Future”, Vaccine (2002) 20:B10-B16.
11. Johnson et al. (1999) Bioorg Med Chem Lett 9:2273-2278.
12. Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491.
13. Pajak, et al., “The Adjuvant OM-174 induces both the migration and maturation of murine dendritic cells in vivo”, Vaccine (2003) 21:836-842.
14. Kandimalla, et al., “Divergent synthetic nucleotide motif recognition pattern: design and development of potent immunomodulatory oligodeoxyribonucleotide agents with distinct cytoline induction profiles”, Nucleic Acids Research (2003) 31(9): 2393-2400.
15. Krieg, “CpG motifs: the active ingredient in bacterial extracts?”, Nature Medicine (2003) 9(7): 831-835.
16. McCluskie, et al., “Parenteral and mucosal prime-boost immunization strategies in mice with hepatitis B surface antigen and CpG DNA”, FEMS Immunology and Medical Microbiology (2002) 32:179-185.
17. Kandimalla, et al., “Toll-like receptor 9: modulation of recognition and cytokine induction by novel synthetic CpG DNAs”, Biochemical Society Transactions (2003) 31 (part 3): 654-658.
18. Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068.
19. Krieg, “Trom A to Z on CpG”, TRENDS in Immunology (2002) 23(2): 64-65.
20. Kandimalla, et al., “Secondary structures in CpG oligonucleotides affect immunostimulatory activity”, BBRC (2003) 306:948-953.
21. Kandimalla, et al., “Toll-like receptor 9: modulation of recognition and cytokine induction by novel synthetic GpG DNAs”, Biochemical Society Transactions (2003) 11(art 3):664-658.
22. Bhagat et al., “CpG penta- and hexadeoxyribonucleotides as potent immunomodulatory agents” BBRC (2003) 300:853-861.
23 Beignon, et al., “The LTR72Mutant of Heat-Labile Enterotoxin of Escherichia coli Enhances the Ability of Peptide Antigens to Elicit CD4+ T Cells and Secrete Gamma Interferon after Coapplication onto Bare Skin”, Infection and Immunity (2002) 10(6):3012-3019.
24 Pizza, et al., “Mucosal vaccines: non toxic derivatives of LT and CT as mucosal adjuvants”, Vaccine (2001) 19:2534-2541.
25 Pizza, et al., “LTK63 and LTR72, two mucosal adjuvants ready for clinical trials” Int. J. Med. Microbiol. (2000) 290(4-5):455-461.
26 Scharton-Kersten et al., “Transcutaneous Immunization with Bacterial ADP-Ribosylating Exotoxins, Subunits and Unrelated Adjuvants”, Infection and Immunity (2000) 68(9):5306-5313.
27 Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280.
28 Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216.
29 Peppoloni et al., “Mutants of the Escherichia coli heat-labile enterotoxin as safe and strong adjuvants for intranasal delivery of vaccines”, Vaccines (2003) 2(2):285-293.
30 Pine et al., (2002) “Intranasal immunization with influenza vaccine and a detoxified mutant of heat labile enterotoxin from Escherichia coli (LTK63)” J. Control Release (2002) 85(1-3):263-270.
31. Singh et al. (2001) J. Cont. Rele. 70:267-276.
32. WO99/27960.
33. WO99/52549.
34. WO01/21207.
35. WO01/21152.
36. Andrianov et al., “Preparation of hydrogel microspheres by coacervation of aqueous polyphophazene solutions”, Biomaterials (1998) 19(1-3):109-115.
37. Payne et al., “Protein Release from Polyphosphazene Matrices”, Adv. Drug. Delivery Review (1998) 31(3):185-196.
38. Stanley, “Imiquimod and the imidazoquinolones: mechanism of action and therapeutic potential” Clin Exp Dermatol (2002) 27(7):571-577.
39. Jones, “Resiquimod 3M”, Curr Opin Investig Drugs (2003) 4(2):214-218.
40. WO99/11241.
41. WO98/57659.
42. European patent applications 0835318, 0735898 and 0761231.

1. An immunogenic composition comprising a combination of GBS polypeptides, said combination consisting of two, three, four or five polypeptides, wherein each polypeptide is encoded by a GBS polynucleotide sequence which is homologous to a polynucleotide sequence of both GAS and Streptococcus pneumoniae.

2. The immunogenic composition of claim 1, wherein said GBS polypeptides are encoded by GBS polynucleotide sequences selected from SEQ ID NOS:17-33, 34-44, 45-61, 62-72, 73-122, 157-167, 180-190, 202-210, 285-295, 385-395, 407-417, 463-474, 508-518, 597-607, 619-629, 641-651, 685-695, 752-762, 823-833, 886-896, 908-918, 930-940, 980-990, 1022-1032, 1132-1142, 1182-1192, 1226-1236, 1248-1258, and 1311-1321.

3. An immunogenic composition comprising a combination of GBS polypeptides, said combination consisting of two, three, four or five polypeptides, wherein each polypeptide is encoded by a GBS polynucleotide sequence which is homologous to a polynucleotide sequence of GAS.

4. The immunogenic composition of claim 3, wherein said GBS polypeptides are encoded by GBS polynucleotide sequences selected from SEQ ID NOS:1-16, 135-145, 223-231, 307-316, 349-357, 430-440, 486-496, 574-584, 729-739, 802-812, 958-968, 1002-1011, 1044-1054, 1066-1076, 1204-1214, and 1271-1281.

5. An immunogenic composition comprising a combination of GBS polypeptides, said combination consisting of two, three, four or five polypeptides, wherein each polypeptide is encoded by a GBS polynucleotide sequence which is homologous to a polynucleotide sequence of Streptococcus pneumoniae.

6. The immunogenic composition of claim 5, wherein said GBS polypeptides are encoded by GBS polynucleotide sequences selected from SEQ ID NOS:327-337, 367-375, 663-673, and 780-790.

7. An immunogenic composition comprising a combination of GBS polypeptides, said combination consisting of two, three, four or five polypeptides, wherein each polypeptide is encoded by a GBS serotype polynucleotide sequence which is homologous to at least one other GBS serotype.

8. The immunogenic composition of claim 2, wherein one or more of the GBS polypeptides are encoded by GBS serotype polynucleotide sequences which are homologous to at least one other GBS serotype.

9. An immunogenic composition comprising a fusion protein, wherein said fusion protein comprises a first polypeptide sequence which is encoded by a GBS serotype polynucleotide which is conserved across one or more GBS serotypes.

10. A polynucleotide sequence, or a fragment comprising at least 10 contiguous polynucleotides, selected from SEQ ID NOS:1-122, 135-145, 157-167, 180-190, 202-210, 223-231, 241-251, 263-273, 285-295, 307-316, 327-337, 349-357, 367-375, 385-395, 407-417, 430-440, 452-457, 463-474, 486-496, 508-518, 530-540, 558-565, 574-584, 597-607, 619-629, 641-651, 663-673, 685-695, 707-717, 729-739, 752-762, 774-776, 780-790, 802-812, 823-833, 846-854, 864-874, 886-896, 908-918, 930-940, 952-954, 958-968, 980-990, 1002-1011, 1022-1032, 1044-1054, 1066-1076, 1088-1098, 1110-1120, 1132-1142, 1154-1156, 1182-1192, 1204-1214, 1226-1236, 1248-1258, 1271-1281, 1293-1301, 1311-1321 1333-1343.

11. The polynucleotide fragment of claim 10, wherein said fragment is derived from a GBS serotype polynucleotide sequence and is homologous to at least one additional GBS serotype polynucleotide sequence.

12. The immunogenic composition of claim 4, wherein one or more of the GBS polypeptides are encoded by GBS serotype polynucleotide sequences which are homologous to at least one other GBS serotype.

13. The immunogenic composition of claim 6, wherein one or more of the GBS polypeptides are encoded by GBS serotype polynucleotide sequences which are homologous to at least one other GBS serotype.
